(19) United States
(12) Patent Application Publication
(10) Pub. No.: US 2004/0241775 A1
(43) Pub. Date: Dec. 2, 2004

(54) Methods and kits for determining risk of pre-term delivery

(75) Inventors: Roberto Romero, Detroit, MI (US); Irina Buhimschi, New Haven, CT (US); Robert Christner, Albuquerque, NM (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(73) Assignee: The Govt. of the USA as represented by the Secretary of the Dept. of Health & Human Services

(21) Appl. No.: 10/713,791

(22) Filed: Nov. 13, 2003

Related U.S. Application Data
(60) Provisional application No. 60/426,096, filed on Nov. 14, 2002.

(51) Int. Cl. G01N 33/53; G01N 33/537; G01N 33/543

(52) U.S. Cl. 435/7.92

(57) ABSTRACT

Biomarkers capable of identifying subjects at risk for preterm complications, such as preterm parturition are disclosed, as well as methods and kits for detecting such biomarkers to identify at risk subjects. A single biomarker or combination of biomarkers comprising a defensin, BPI, calprotectin, or a calgranulin are used to qualify the risk of preterm complications in a subject. In particular, the concentrations of the biomarkers correlate with the magnitude of preterm complications and can diagnose or prognose specific preterm complications, such as preterm parturition, preterm premature rupture of the membranes (PROM), intra-amniotic inflammation, and/or microbial invasion of the amniotic cavity (MIAC).

### CROSS REFERENCE TO RELATED APPLICATION

This disclosure claims priority to U.S. Provisional Patent Application No. 60/426,096, filed Nov. 14, 2002, which is incorporated by reference herein.

### BACKGROUND OF THE INVENTION

Premature birth is the leading cause of perinatal morbidity and mortality. Every year approximately 4.5 million premature babies are born worldwide, and, despite considerable advances in neonatal care, their mortality rate remains high. Moreover, survivors are at risk for long-term disabilities, including developmental delay, cerebral palsy, blindness, deafness, and chronic lung disease. Thus, the prevention of prematurity is an important challenge to obstetrics and perinatal medicine. The limited success in preventing prematurity has been attributed, in part, to the fact that premature parturition is a syndrome caused by multiple pathological processes such as infection, vascular disease, uterine over-distension, and chronic stress.

Intrauterine infection has emerged as a common and important cause of preterm delivery, as at least a third of all preterm births occur to mothers with microbial invasion of the amniotic cavity. Intrauterine infection often results in fetal infection with the development of the fetal inflammatory response syndrome, a risk factor for the impending onset of labor, short-term neonatal complications, and long-term handicaps, such as cerebral palsy and chronic lung disease.

Despite the compelling evidence that infection is often causally linked to preterm delivery, antibiotic treatment of subjects with premature labor has not proven effective in preventing preterm delivery or neonatal morbidity in most trials. A potential explanation is that many subjects presenting with preterm labor do not have intrauterine infection and therefore may not benefit from antimicrobial therapy. Moreover, intra-amniotic and fetal inflammation per se are linked to adverse outcome, even in the absence of microbiologically proven infection. Thus, the accurate and rapid identification of a pregnant woman with subclinical intrauterine inflammation is an urgent priority for the development of rational therapy.

Current evidence indicates that analysis of amniotic fluid, which is normally sterile, is the most accurate means of determining the presence or absence of infection and/or inflammation. Available tests have limited sensitivity and specificity, however, and the results of standard microbiologic techniques, such as microbial culture, take time and are not available for immediate management decisions.

### SUMMARY

To address these and other needs, disclosed herein are methods of determining whether a subject is at risk for preterm complications such as preterm parturition, preterm premature rupture of the membranes (PROM), intra-amniotic inflammation, and/or microbial invasion of the amniotic cavity (MIAC), by measuring levels of one or more biomarkers including defensins, bactericidal/permeability-increasing protein (BPI), calprotectin, and calgranulin, such as calgranulin A, B, or C. Also disclosed are kits for performing such methods.

Determining the subject's risk of preterm complications in some embodiments includes analyzing a sample of amniotic fluid from the subject for a level of a single biomarker or a combination of biomarkers. In some cases, the sample is analyzed to detect at least one calgranulin or a calprotectin complex. In other cases, the sample also is analyzed to detect at least one defensin. In specific embodiments the calgranulin is calgranulin A or calgranulin C and the defensin is HNP-1 (alpha-defensin 1) or HNP-2 (alpha-defensin 2). In some embodiments the sample is analyzed to detect a combination of BPI, calprotectin, and defensin. Elevated levels of these biomarkers or combinations of biomarkers indicate a risk of pre-term complications.

In some embodiments of the disclosed methods a spectrum is generated by mass spectroscopic analysis of a sample of the subject's amniotic fluid. The mass spectroscopic analysis can include profiling on a biologically- or chemically-derivatized affinity surface. The subject's risk of pre-term complications is determined by putting the spectrum through pattern-recognition analysis that is keyed to at least one peak indicative of the presence of a biomarker, such as calgranulin in the sample. The pattern-recognition analysis additionally is keyed to at least one peak indicative of a defensin in some cases. In specific embodiments, the pattern-recognition analysis is keyed to at least one of calgranulin A or calgranulin C and at least one of HNP-1 (alpha-defensin 1) or HNP-2 (alpha-defensin 2).

One embodiment of the disclosed kit includes an adsorbent that binds to at least one of the disclosed biomarkers such as an antibody immobilized on a solid substrate. The kit may be an ELISA in which an enzyme-antibody conjugate used to detect biomarker immobilized on the solid substrate. In some embodiments, the adsorbent, such as a hydrophobic adsorbent, is immobilized on a probe and the biomarker is detected by laser desorption/ionization mass spectrometry.

### BRIEF DESCRIPTION OF THE DRAWINGS

FIG. 1 is a flow chart of the distribution of subjects used for “learning” Surface Enhanced Laser Desorption and Ionization (SELDI) profiles in amniotic fluid.

FIG. 2 presents representative protein mass-spectral profiles of “diseased,” “non-diseased,” and T-CRL subjects who were studied during the learning phase. The discriminatory peaks composing the M (P1-P13) and MR score (circled peaks) are shown within the three molecular weight areas of interest: 3300-3500 dalton (Da) under the CHCA-LL experimental protocol (a); 3500-3800 Da under CHCA-HL protocol (b); and 10-14 kDa under the SPA protocol (c).

FIG. 3 depicts subject data as follows: (a) Scattergrams represent the relationship between MR score of the amniotic fluid samples obtained from preterm subjects (n=77) and the state of intra-amniotic inflammation (WBC>100 cells/mm³) or intra-amniotic infection (positive amniotic fluid culture result). Closed circles represent the “diseased” subjects. Open diamonds denote the “non-diseased” group. The rest of the subjects are shown with open circles. (b) Survival analysis of the percent of undelivered subjects after amniocentesis was performed in the preterm group of subjects. Closed squares represent subjects with MR scores of 3 or 4. Open squares are subjects with MR scores of 0-2.

FIG. 4 presents data that identify peaks P1, P2, and P3 as neutrophil defensins (HNP-1-3) (a) and peaks P7 and P8 as calgranulins (b) based by on-chip antibody capture assays. Antibody-specific peaks are distinguished at the same mass with profiling tracings (H4), on spots where the antibody (Ab) has been pre-adsorbed but not on the spots pre-treated with 1 gG. The amniotic fluid samples were from representative “diseased” and “non-diseased” subjects. Samples also were loaded onto Tricine gels and either stained with Coomasie blue (b) or processed for Western blotting, using the same antibody utilized for antibody capture: anti HNP (insert at a) or Mac 387(c).

FIG. 5 presents a quantitative analysis of the peaks composing the MR score (log of normalized peak intensity) in the cohort of preterm subjects (n=77), grouped by the presence or absence of intra-amniotic inflammation (+WBC: WBC>100/mm³) or microbiologically proven infection (+AFC: positive amniotic fluid culture results). The lines represent the means of the groups.

FIG. 6 depicts a quantitative analysis of a mixture of equal amounts of recombinant HNP-1 and HNP-2 on H4 spots. (a) SELDI profiles obtained after application of 1 μg (above) or 2 ng (below) of HNP 1-2 mixture. (b) Log normalized peak intensity of the SELDI tracings versus the amount of HNP-1-2 mixture spotted. Each point represents the mean and SD from three individual spots. (c) Amniotic fluid HNP-1-3 concentration as measured by ELISA in the cohort of preterm subjects (n=77) grouped by the presence or absence of intra-amniotic inflammation (+WBC: WBC>100/mm³) or microbiologically proven infection (+AFC: positive amniotic fluid culture results). The lines represent the means of the groups. (d) Correlation between the HNP-1-3 amount in amniotic fluid measured by ELISA (log) and log normalized intensity of the P2 peak (HNP-1) by SELDI.

FIG. 7 is three graphs showing the amniotic fluid concentration of bactericidal/permeability-increasing protein (BPI), human neutrophil defensins (HNP) 1-3 and calprotectin in women in the mid-trimester who delivered a normal neonate at term and in women at term without labor. Dotted lines indicate detection limits: (a) 0.07 ng/ml; (b) 0.02 ng/ml; (c) 0.007 mg/ml; *, statistically significant.

FIG. 8 is three graphs showing amniotic fluid concentration of bactericidal/permeability-increasing protein (BPI), human neutrophil defensins (HNP) 1-3 and calprotectin in women with preterm labor and intact membranes. Dotted lines indicate detection limits: (a) 0.07 ng/ml; (b) 0.02 ng/ml; (c) 0.007 mg/ml; *, statistically significant.

FIG. 9 is three graphs showing amniotic fluid concentration of bactericidal/permeability-increasing protein (BPI), human neutrophil defensins (HNP) 1-3 and calprotectin in women at term with and without labor and without microbial invasion of the amniotic cavity (MIAC). Dotted lines indicate detection limits: (a) 0.07 ng/ml; (b) 0.02 ng/ml; (c) 0.007 mg/ml; *, statistically significant.

FIG. 10 is three graphs showing amniotic fluid concentration of bactericidal/permeability-increasing protein (BPI), human neutrophil defensins (HNP) 1-3 and calprotectin in women with pretermpremature rupture of membranes (PROM) with and without microbial invasion of the amniotic cavity (MIAC). Dotted lines indicate detection limits: (a) 0.07 ng/ml; (b) 0.02 ng/ml; (c) 0.007 mg/ml; *, statistically significant.

FIG. 11 is three graphs showing amniotic fluid concentration of bactericidal permeability-increasing protein (BPI), human neutrophil defensins (HNP) 1-3 and calprotectin in women with preterm labor and term delivery and women with preterm PROM in the absence of microbial invasion of the amniotic cavity (MIAC). Dotted lines indicate detection limits: (a) 0.07 ng/ml; (b) 0.02 ng/ml; (c) 0.007 mg/ml; *statistically significant.

FIG. 12 is two graphs showing receiver-operating characteristic (ROC) curves of bactericidal/permeability-increasing protein (BPI), human neutrophil defensins (HNP) 1-3, calprotectin and the combined markers (composite marker) in women with preterm labor and intact membranes. (a) ROC curve for the identification of positive amniotic fluid culture for micro-organisms. Areas under the curve for amniotic fluid BPI, 0.87; amniotic fluid HNP 1-3, 0.85; amniotic fluid calprotectin, 0.84, and composite marker, 0.84 (p<0.001 for all). (b) ROC curve for the identification of intra-amniotic inflammation (defined as amniotic fluid white blood cell count ³ 50 cells/ml). Areas under the curve for amniotic fluid BPI, 0.96; amniotic fluid HNP 1-3, 0.95; amniotic fluid calprotectin, 0.94; and composite marker, 0.94 (p<0.001 for all).

FIG. 13 is four graphs showing the amniocentesis-to-delivery interval according to the amniotic fluid concentrations of bactericidal permeability-increasing protein (BPI), human neutrophil defensins (HNP) 1-3, calprotectin and the composite marker. Subjects with amniotic fluid concentrations of BPI≧15.5 ng/ml, HNP 1-3≧53.2 ng/ml and calprotectin≧48.4 mg/ml had a significantly shorter amniocentesisto-delivery interval than subjects below those cut-off values.

### DETAILED DESCRIPTION OF THE PREFERRED EMBODIMENTS

The term “subject” refers to an animal, such as a pregnant mammal, for example a pregnant human.

The term “preterm complications” refers to pre-term parturition, preterm premature rupture of the membrane (PROM), intra-amniotic inflammation, and/or microbial invasion of the amniotic cavity (MIAC).

The term “preterm parturition” refers to delivery of offspring prior to the expiration of the normal gestational period, in humans before about the 38^{th} week of gestation.

The term “preterm PROM” or “preterm Premature Rupture of Membranes” refers to rupture of the amniotic sac prior to the onset of labor contractions and before the expiration of the normal gestational period.

Defensins, proteins of the innate immune system, are also known as human neutrophil peptides 1-3 (HNP 1-3). HNP-1,-2 and -3 belong to the family of cationic, trifsulfide-containing microbicidal peptides. Their production and release are induced by cytokines and microbial products such as lipopolysaccharide, a component of the cell wall of Gram negative bacteria.

Calprotectin is a calcium binding protein also called calcium binding leukocyte L1 protein and cystic fibrosis antigen. It belongs to the S100 family of calcium binding proteins and is a complex of 8- and 14-kDa subunits, known as calgranulin A and calgranulin B, respectively. It is known to have antimicrobial action.

Calgranulins are also members of the S100 family of proteins with antimicrobial action. Calgranulin A (s100A8) is a 10.834 kDa peptide also known as MRP8. Calgranulin B (S100A9) is a 13.242 kDa peptide also known as MRP14. Calgranulin A and B can combine to form homodimers and heterodimers, such as calprotectin, which also have antimicrobial properties. Calgranulin C (S100A12) is a 10.444 kDa peptide.

Bactericidal/permeability-increasing protein (BPI) is a 55 kDa protein that binds to lipopolysaccharides (LPS), and exerts bacteriostatic and bactericidal effects against a wide variety of Gram-negative bacterial species.

Biomarkers are disclosed herein, the presence of which in the amniotic fluid indicates that the subject is at risk for pre-term complications, such as preterm parturition. In the present context, a “biomarker” is an organic biomolecule, particularly a polypeptide or protein, which is differentially present in a sample of amniotic fluid taken from a subject at-risk for preterm complications. For example, a subject having intra-amniotic inflammation, as compared to a similar sample taken from a “normal” subject that did not experience pre-term parturition subsequent to sampling. A biomarker is differentially present in samples from a normal subject and one at-risk for preterm parturition, respectively, if it is present at an elevated level or a decreased level in latter samples as compared to samples of normal subjects. In some embodiments the biomarker or biomarkers are antimicrobial peptides/proteins.

The disclosed biomarkers include defensins, such as defensin 1, 2, and 3, BPI, calprotectin, and calgranulins, such as calgranulin A, B, and C.

“Diagnostic concentrations or levels” refers to a concentration (for example of a biomarker in amniotic fluid) that provides clinical information to detect a pathological condition or predict its course. An example of a diagnostic concentration is a concentration of calprotectin or calgranulin that differentiates probable normal parturition from probable pre-term parturition. Another example of a diagnostic concentration is one that detects the presence of probable intra-amniotic inflammation.

In general, the disclosed methods include detecting whether the disclosed biomarkers (or any one or more of them) are present in the subject's amniotic fluid. Any method for detecting the levels of the biomarkers in the subject's amniotic fluid can be used. In particular embodiments, a sample amniotic fluid from the subject is analyzed to determine the presence and concentration of one or more of the disclosed biomarkers.

The sample of amniotic fluid in some embodiments is obtained by amniocentesis. Amniocentesis is performed, for example, by inserting a needle with a stylet (such as a 20-21 gauge needle) guided by an imaging device (such as ultrasound) through the abdominal wall of the subject and into the amniotic cavity. The stylet is removed and a sample of amniotic fluid (for example about 20 ml) is aspirated. To lessen the chance of maternal contamination, the initial 2 ml or so can be discarded.

Detecting the levels of the biomarkers includes the use of any method that identifies the presence of the biomarker(s) and can quantify the amount of biomarker(s) present. Such methods can be performed with specific binding agents, for example antibodies, such as monoclonal antibodies, that recognize the biomarker, as in an ELISA. Examples of specific binding agents are discussed in Example 2. Such methods also include gas phase ion spectrometry (for example, mass spectrometry). Other detection methods employed to this end include optical methods, electrochemical methods (voltametry and amperometry techniques), atomic force microscopy, and radio frequency methods (e.g., multipolar resonance spectroscopy). Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry). Immunoassays in various formats, such as ELISA, likewise can be adapted for detection of biomarkers, for example by using antibiodies to the biomarkers.

In some embodiments the detection of biomarkers entails contacting a sample of amniotic fluid from the subject with a substrate, having an adsorbent thereon, under conditions that allow binding between the biomarker and the adsorbent, and then detecting the biomarkers with gas phase ion spectrometry (for example, mass spectrometry).

In some embodiments using mass spectroscopy for detection of the biomarkers Surface Enhanced Laser Desorption and Ionization (SELDI) is used, as described, for example, in U.S. Pat. Nos. 5,719,060 and 6,225,047, both to Hutchens and Yip. With this SELDI technique, the surface of a probe that presents the analyte (here, one or more of the biomarkers) to the energy source plays an active role in desorption/ionization of analyte molecules. In this context, “probe” refers to a device adapted to engage a probe interface and to present an analyte to ionizing energy for ionization and introduction into a gas phase ion spectrometer, such as a mass spectrometer. A probe typically includes a solid substrate, either flexible or rigid, with a sample-presenting surface, on which an analyte is presented to the source of ionizing energy.

One version of SELDI, called “Surface-Enhanced Affinity Capture” or “SEAC,” involves the use of probes comprised of a chemically selective surface (“SELDI probe”). A “chemically selective surface” is one to which is bound either the adsorbent, also called a “binding moiety” or “capture reagent,” or a reactive moiety capable of binding a capture reagent (e.g., through a reaction forming a covalent or coordinate covalent bond).

The phrase “reactive moiety” here denotes a chemical moiety capable of binding a capture reagent. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine-containing peptides. A “reactive surface” is a surface to which a reactive moiety is bound. An “adsorbent” or “capture reagent” can be any material capable of binding a biomarker of the invention. Suitable adsorbents for use in SELDI, according to the invention, are described in U.S. Pat. No. 6,225,047, supra.

One type of adsorbent is a “chromatographic adsorbent,” which is a material typically used in chromatography. Chromatographic adsorbents include, for example, ion exchange materials, metal chelators, immobilized metal chelates, hydrophobic interaction adsorbents, hydrophilic interaction adsorbents, dyes, mixed mode adsorbents (e.g., hydrophobic attraction/electrostatic repulsion adsorbents). “Biospecific adsorbent” is another category containing adsorbents that contain a biomolecule, for example, a nucleotide, a nucleic acid molecule, an amino acid, a polypeptide, a simple sugar, a polysaccharide, a fatty acid, a lipid, a steroid or a conjugate of these (e.g., a glycoprotein, a lipoprotein, a glycolipid). In certain instances, the biospecific adsorbent can be a macromolecular structure such as a multiprotein complex, a biological membrane, or a virus. Illustrative biospecific adsorbents are antibodies, receptor proteins, and nucleic acids. A biospecific adsorbent typically has higher specificity for a target analyte than a chromatographic adsorbent.

Another version of SELDI is Surface-Enhanced Neat Desorption (SEND), which involves the use of probes comprising energy absorbing moleculese chemically bound to the probe surface (“SEND probe”). The phrase “Energy absorbing molecules” (EAM) denotes molecules capable of absorbing energy from a laser desorption ionization source and, thereafter, contributing to desorption and ionization of analyte molecules in contact therewith. The EAM category includes molecules used in MALDI frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. The category also includes EAMs used in SELDI, as enumerated, for example, by U.S. Pat. No. 5,719,060 and U.S. Ser. No. 60/351,971, filed Jan. 25, 2002.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light (e.g., to laser light). For instance, see U.S. Pat. No. 5,719,060. SEPAR and other forms of SELDI are readily adapted to detecting a biomarker or biomarker profile, pursuant to the present invention.

The detection of the biomarkers can be enhanced by using certain selectivity conditions, such as adsorbents or washing solutions. The phrase “wash solution” refers to an agent, typically a solution, used to affect or modify adsorption of an analyte to an adsorbent surface and/or remove unbound materials from the surface. The elution characteristics of a wash solution can depend, for example, on pH, ionic strength, hydrophobicity, degree of chaotropism, detergent strength, and temperature.

In some cases a sample is analyzed by means of a “biochip,” a term denoting a solid substrate that has a generally planar surface, to which a capture reagent (adsorbent) is attached. Frequently, the surface of the biochip comprises a plurality of addressable locations, each having the capture reagent bound there. The biochip in some embodiments is adapted to engage a probe interface and, hence, function as a probe which can be inserted into a gas phase ion spectrometer, preferably a mass spectrometer. Alternatively, in other cases the biochip is mounted onto another substrate to form a probe that can be inserted into the spectrometer.

A variety of biochips are available for the capture of biomarkers from commercial sources such as Ciphergen Biosystems (Fremont, Calif.), Packard BioScience Company (Meriden, Conn.), Zyomyx (Hayward, Calif.), and Phylos (Lexington, Mass.). Examples of these biochips are those described in U.S. Pat. No. 6,225,047, supra, and U.S. Pat. No. 6,329,209 (Wagner et al.), and in PCT publications WO 99/51773 (Kuimelis and Wagner) and WO 00/56934 (Englert et al.).

More specifically, biochips produced by Ciphergen Biosystems have surfaces, presented on an aluminum substrate in strip form, to which are attached, at addressable locations, chromatographic or biospecific adsorbents. The surface of the strip is coated with silicon dioxide.

Illustrative of Ciphergen ProteinChip® arrays are biochips H4, SAX-2, WCX-2, and IMAC-3, which include a functionalized, cross-linked polymer in the form of a hydrogel physically attached to the surface of the biochip or covalently attached through a silane to the surface of the biochip. The H4 biochip has isopropyl functionalities for hydrophobic binding. The SAX-2 biochip has quaternary ammonium functionalities for anion exchange. The WCX-2 biochip has carboxylate functionalities for cation exchange. The IMAC-3 biochip has nitriloacetic acid functionalities that adsorb transition metal ions, such as Cu⁺⁺ and Ni⁺⁺ by chelation. These immobilized metal ions, in turn, allow for adsorption of biomarkers by coordinate bonding. In keeping with the principles described above, a substrate with an adsorbent is contacted with the sample, containing amniotic fluid, for a period of time sufficient to allow biomarker that may be present to bind to the adsorbent. After the incubation period, the substrate is washed to remove unbound material. Any suitable washing solutions can be used; preferably, aqueous solutions are employed.

An energy absorbing molecule then is applied to the substrate with the bound biomarkers. As noted, this type of molecule is one that absorbs energy from an energy source in a gas phase ion spectrometer, thereby assisting in desorption of biomarkers from the substrate. Examples of energy absorbing molecules include, as noted above, cinnamic acid derivatives, sinapinic acid, and dihydroxybenzoic acid. Preferably sinapinic acid is used.

The biomarkers bound to the substrates are detected in a gas phase ion spectrometer, as noted above. The biomarkers are ionized by an ionization source such as a laser, the generated ions are collected by an ion optic assembly, and then a mass analyzer disperses and analyzes the passing ions. The detector then translates information of the detected ions into mass-to-charge ratios, which are represented as signals of varying intensity Thus, both the quantity and mass of the biomarker can be determined.

Data generated by desorption and detection of the disclosed biomarkers can be analyzed with the use of a programmable digital computer. The computer program analyzes the data to indicate the number of markers detected, and optionally the strength of the signal and the determined molecular mass for each biomarker detected. Data analysis can include steps of determining signal strength of a biomarker and removing data deviating from a predetermined statistical distribution. For example, the observed peaks can be normalized by calculating the height of each peak relative to some reference. The reference can be background noise generated by the instrument and chemicals such as the energy absorbing molecule which is set as zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Software used to analyze the data can include a code that applies an algorithm to the analysis of the signal in order to determine whether the signal represents a peak in a signal that corresponds to a biomarker according to the present invention. The software also can subject the data regarding observed biomarker peaks to classification tree or ANN analysis, to determine whether a biomarker peak or a combination of biomarker peaks is present, indicating a diagnosis of a preterm complication.

In some embodiments the subject at risk for preterm complications is identified by detecting a single biomarker, such as a calgranulin (for example calgranulin C) or calprotectin. In other embodiments the subject at risk for preterm complications is identified by detecting a combination of biomarkers (“a biomarker profile”). In certain instances the biomarker profile includes calgranulin, such as calgranulin C, and any other biomarker. In other cases the biomarker profile includes defensins 1-3, BPI, and calprotectin.

In some embodiments the subject is at risk for preterm complications if concentrations of the disclosed biomarkers are detected in the subject's amniotic fluid that are at or above certain cut-off or diagnostic concentrations for the corresponding biomarker. The cut-off concentration for calprotectin is about 12 ng/ml or higher, such as about 13, 14, 15, 16, or 17 ng/ml, for example about 15.3 ng/ml. The cut-off concentration for BPI is about 2 ng/ml or higher, such as about 2, 2.5, 3, 3.5,4, 4.5, 5, 5.5, or 6 ng/ml or higher, for example 2.03 ng/ml. The cut-off concentration for defensins is about 2 ng/ml, for example about 2.5, 3, 3.5, 4, 4.5, 5, 5.5, or 6 ng/ml or higher, such as about 2.93 ng/ml. In other embodiments, the subject is at risk for preterm complications if concentrations of two or more of the disclosed biomarkers are detected in the subject's amniotic fluid (for example calprotectin and defensin) that are at or above the cut-off concentrations disclosed above in this paragraph.

In still other embodiments, the subject is at risk for a specific preterm complication if concentrations of the disclosed biomarkers are detected in the subject's amniotic fluid that are at or above complication specific cut-off concentrations. Examples of such complication or outcome specific concentrations are disclosed in Example 2, for example in Tables 6-9.

The complication specific diagnostic concentration for pre-term delivery for calprotectin is about 12 ng/ml or higher, such as about 13, 14, 15, 16, or 17 ng/ml, for example about 15.3 ng/ml. For BRI the diagnostic concentration is about 2 ng/ml or higher, such as about 2.5, 3, 3.5, 4, 4.5, 5, 5.5, or 6 ng/ml or higher, for example 2.03 ng/ml. For defensins the diagnostic concentration is about 2 ng/ml, for example about 2.5, 3, 3.5, 4, 4.5, 5, 5.5, or 6 ng/ml or higher, such as about 2.93 ng/ml.

The complication specific diagnostic concentration for MIAC for calprotectin is about 20 ng/ml or higher, such as about 24, 26, or 28 ng/ml, for example about 27.9 ng/ml. For BPI the diagnostic concentration is about 3 ng/ml or higher, such as about 3.5, 4, 4.5, 5, 5.5, or 6 ng/ml or higher, for example 3.7 ng/ml. For defensins the diagnostic concentration is about 6 ng/ml or higher, for example about 6.5, 7, 7.5, 8, 8.5, or 9 ng/ml or higher, such as about 7.8 ng/ml.

The complication specific diagnostic concentration for preterm PROM for calprotectin is about 12 ng/ml, foe example about 13, 14, 15, 16, or 17 ng/ml or higher, such as about 15.3 ng/ml. For BPI the diagnostic concentration is about 2 ng/ml or higher, such as about 2.5, 3, 3.5, 4, or 4.6 ng/ml or higher, such as about 2.03 ng/ml. For defensins the diagnostic concentration is about 2 ng/ml, for example about 2.5, 3, 3.5, 4, 4.5, 5, 5.5, or 6 ng/ml or higher, such as about 2.92 ng/ml.

The complication specific diagnostic concentration for the combination of preterm PROM and MIAC for calprotectin is about is about 25 ng/ml or higher, such as about 26, 27, 28, 29, 30, 31, 32, or 33 ng/ml or higher, for example about 32.66 ng/ml. For BPI the diagnostic concentration is about 28 ng/ml or higher, for example about 29, 30, 31,32, 33, or 34 ng/ml or higher, such as about 32.66 ng/ml. For defensins the diagnostic concentration is about 65 ng/ml or higher, for example about 67, 69, 71, 73, 75, 77, or 79 ng/ml or higher, such as about 78.78 ng/ml.

The complication specific cut-off for intra-amniotic inflammation for calprotectin is about 40 ng/ml or higher, for example about 42, 44, 46, 48, or 50 ng/ml, such as about 48.4 ng/ml. For BPI the cut-off is about 12 ng/ml or higher, such as about 13, 14, 15, or 16 ng.ml, for example about 15.5 ng/ml. For defensins the cut-off is about 45 ng/ml or higher, for example about 47, 49, 51, 53, or 55 ng/ml or higher, such as about 53.2 ng/ml.

In other embodiments, the subject is at risk for a specific preterm complication if if concentrations of two or more of the disclosed biomarkers (for example calprotectin and BPI) are detected in the subject's amniotic fluid that are at or above the diagnostic concentration concentrations disclosed above corresponding to the specific preterm complication. The disclosed methods provide a rapid and reliable proteomic approach to identifying subjects at risk for preterm complications including pre-term parturition. This is the first proteomic characterization of amniotic fluid in premature labor, and detailed analyses of the biomarkers, permits characterization, and quantitative validation of the changes involved. In particular, the concentrations of the biomarkers correlate with the magnitude of the biological phenomena of interest, such as intra-amniotic inflammation and preterm delivery.

Moreover, in some embodiments the protein detection or proteomic analysis is combined with conventional or molecular microbiological techniques to detect microorganisms that may be responsible for the preterm complications that are suggested by the detection of the biomarkers, thereby informing selection of an antimicrobial therapy. For example, an elevation of the biomarker can be a screening tool that indicates subsequent microbiological analysis should be performed to identify and treat a particular pathogen.

The disclosed methods of proteomic analysis of amniotic fluid, provide semi-quantitative information that correlates with the magnitude of inflammation, as determined by the intensity of intrauterine inflammation (i.e., correlation with white blood cell counts) and the clinical outcome (i.e., relationship between duration of pregnancy and MR score, described below). In particular, the disclosed methods provide a means for predicting preterm delivery, based on an analysis of patterns of particular biomarkers.

Kits aiding in the diagnosis of preterm complications, such as intra-amniotic inflammation, by detecting biomarkers are also disclosed. The kits screen for the presence of biomarkers and combinations of biomarkers that are differentially present in samples from subjects at risk for preterm complications. Such kits may include reagents for detecting the biomarkers, such as a specific binding agent or agents for detecting and/or quantifying one or more of the biomarkers. In particular examples, the kits also include instructions for using the reagents to detect and/or quantitate the biomarkers, and/or quantitating the presence of one or more of the biomarkers to establish a likelihood of preterm complications, such as PROM or premature delivery prior to the end of normal gestation.

One embodiment of the disclosed kits comprises a substrate having an adsorbent thereon, wherein the adsorbent is suitable for binding a biomarker according to the invention, and a washing solution or instructions for making a washing solution, in which the combination of the adsorbent and the washing solution allows detection of the biomarker using gas phase ion spectrometry. In certain cases, such a kit comprises an immobilized metal affinity capture chip, such as the H4 chip.

Another embodiment of the disclosed kits includes a first substrate, comprising an adsorbent thereon, and a second substrate onto which the first substrate is positioned to form a probe, which can be inserted into a gas phase ion spectrometer. Another embodiment of the disclosed kits comprises a single substrate that can be inserted into the spectrometer.

Some embodiments of the disclosed kits, include instructions for suitable operational parameters in the form of a label or separate insert. For example, the instructions may inform a consumer how to collect the sample or how to wash the probe.

The disclosed biomarkers also are useful in the production of other diagnostic assays for detecting the presence of the biomarker in a sample. For example, such assays may comprise, as the “adsorbent,” “binding moiety,” or “capture reagent,” an antibody to one or more of the biomarkers, with the proviso that at least one of the biomarkers is a calgranulin. The antibody is mixed with a sample suspected of containing the biomarkers and monitored for biomarker-antibody binding. The biomarker antibody is identified with a radioactive or enzyme label. In a preferred embodiment, the biomarker antibody is immobilized on a solid matrix such that the biomarker antibody is accessible to biomarker in the sample. The sample then is brought into contact with the surface of the matrix, and the surface is monitored for biomarker-antibody binding.

For example, the biomarker can be detected in an enzyme-linked immunosorbent assay (ELISA), in which biomarker antibody is bound to a solid phase and an enzyme-antibody conjugate is employed to detect and/or quantify biomarker present in a sample. Alternatively, a western blot assay can be used in which solubilized and separated biomarker is bound to nitrocellulose paper. The combination of a highly specific, stable liquid conjugate with a sensitive chromogenic substrate allows rapid and accurate identification of samples. All solutions required for blocking and washing the membrane and for diluting antibodies are provided. The biomarker is detected by an enzyme or label-conjugated anti-immunoglobulin (1 g), such as horseradish peroxidase-1 g conjugate, by incubating the filter paper in the presence of a precipitable or detectable substrate. Western blot assays have the advantage of not requiring purity greater than 50% for the desired biomarker(s). Descriptions of ELISA and western blot techniques are found in Chapters 10 and 11 of Ausubel et al. (eds.), CURRENT PROTOCOLS IN MOLECULAR BIOLOGY (John Wiley and Sons, 1988).

### EXAMPLE 1

This example illustrates that detection of the disclosed biomarkers calgranulin and defensin in a sample of amniotic fluid from a subject indicates a diagnosis of preterm complications, such as preterm parturition and intra-amniotic inflammation.

The disclosed biomarkers in this example were identified by comparing mass spectra of samples derived from amniotic fluid from two groups of pregnant subjects: 1) subjects with intra-amniotic inflammation, and 2) normal subjects. The subjects were diagnosed according to standard clinical criteria.

These two pools were used in a wide range of dilutions to test various chip surfaces, produced by Ciphergen Biosystems (Fremont, Calif.), for optimal discriminatory performance, including: reverse phase H4, a hydrophobic surface with C-16 long chain aliphatic residues; SAX 2, a strong anion exchanger; WCX2, a quaternary ammonium, weak cation exchanger; IMAC, carboxylate residues; metal affinity. For H4 chip surfaces, optimization involved additional hydrophobic washes of acetonitrile gradients (10% to 75%). A procedure where 2 ul of amniotic fluid, diluted 10-fold in phosphate buffer saline (PBS), was placed on a spot of a 24-spot H4 array and incubated in a humidified box, thus avoiding desiccation, was found to be optimal for individual peak detection and low signal-to-noise (S/N) ratios. As described in greater detail below, however, other chips can be used, so long as they have binding characteristics suitable for binding the disclosed biomarkers.

### Study Population

A total of 114 amniotic fluid samples from distinct subjects were analyzed in this study. Ninety samples were used to establish proteomic patterns with diagnostic value, and 24 samples were used to test and validate the algorithm. Amniotic fluid was obtained by amniocentesis performed for the assessment of the microbiological status of amniotic cavity and/or fetal lung maturity. Samples from subjects at term were obtained at the time of elective caesarian section. Preterm labor was defined as the presence of uterine contractions (at least 3 in 10 minutes (min)) or advanced cervical dilatation at less than 37 weeks of gestation. PROM was diagnosed by sterile speculum examination confirming leakage of amniotic fluid in the vagina, and positive ferning and nitrazine test results. Samples for research were collected under IRB protocols approved by Wayne State University after written informed consent was obtained. The utilization of these samples for research was approved by the IRB of the National Institute of Child Health and Human Development. Cultures for aerobic, anaerobic bacteria and genital Mycoplasmas, Gram stain, counts of white and red blood cells were performed immediately after collection. The remaining amniotic fluid was centrifuged at 700 g. 4° C. for 10 min., and was then stored in aliquots at −80° C. until analysis.

### Protein Profiling Protocol for SELDI-TOF Mass Spectrometry

Preliminary experiments were conducted to optimize the protein profiling protocol. Two pools of amniotic fluid were generated, using samples from two extreme “diseased” and “non-diseased” groups, as shown in Table 1, below.

These two pools were used in a wide range of dilutions to test various chip surfaces for optimal discriminatory performance (reverse phase H4: hydrophobic surface with C-16 long chain aliphatic residues; strong anion exchanger SAX2: quaternary ammonium; weak cation exchanger WCX2: carboxylate residues; metal affinity: IMAC). For H4 chip surfaces, optimization involved additional hydrophobic washes of acetonitrile gradients (10% to 75%). A procedure was developed where 2 μl of amniotic fluid diluted 10-fold in phosphate buffer saline (PBS) was placed on a spot of a 24-spot H4 array, a product of Ciphergen Biosystems (Fremont, Calif.), and incubated in a humidified box to avoid desiccation was optimal for individual peak detection and low signal-to-noise (S/N) ratios. After one hour, the sample was aspirated and the spots washed individually with 3 volumes of 5 μl 25% aqueous acetonitrile solution, left to air dry and then overlaid with matrix solution (energy absorbing molecule) diluted in 0.5% trifluoroacetic acid/50% acetonitrile.

TABLE 1
Subject chart data and amniotic fluid analysis of preterm subjects used for
“learning” SELDI profiles

| | PT-CRL | | | | |
|---|---|---|---|---|---|
| | “non- | +AFC+WBC | | | |
| Subject | diseased” | “diseased” | −AFC+WBC | +AFC−WBC | −AFC−WBC |
| Characteristics | n = 17 | n = 21 | n = 7 | n = 8 | n = 24 |
| Age (years: mean ± SD) | 27 ± 7 | 27 ± 7 | 24 ± 5 | 28 ± 4 | 24 ± 5 |
| Ethnicity (% | 82% | 85.7% | 85.7% | 100% | 96% |
| African-American) | | | | | |
| Parity (median | 1 [0-2] | 1 [0-7] | 1 [0-6] | 2 [0-6] | 1 [0-8] |
| [range]) | | | | | |
| Gravidity (median | 4 [3-10] | 3 [1-10] | 5 [1-7] | 5.5 [1.8] | 2 [1-11] |
| [range]) | | | | | |
| Subjects with | 3/12 | 3/11 | 3/5 | 3/7 | 6/13 |
| history of preterm | | | | | |
| delivery/ | | | | | |
| multiparours | | | | | |
| subjects | | | | | |
| Admission- | | | | | |
| Amniocentesis- | | | | | |
| Delivery | | | | | |
| GA at | 29.7 ± 3 | 27.5 ± 4 | 26.5 ± 2 | 32.1 ± 1 | 30.0 ± 3 |
| amniocentesis (wks ± SD) | | | | | |
| GA at delivery | 38.5 ± 1 | 27.6 ± 4 | 27.7 ± 3 | 33.0 ± 1 | 31.8 ± 4 |
| (wks ± SD) | | | | | |
| PPROM (n [%]) | 0 | 8 [38%] | 4 [57%] | 7 [87.5%] | 8 [33%] |
| Induced/ | 2/15 | 8/11 | 0/6 | 5/3 | 8/16 |
| spontaneous labor | | | | | |
| Cesarean deliveries | 1 | 4 | 1 | 0 | 2 |
| Birth weight (grams ± SD) | 3290 ± 491 | 1117 ± 589 | 1073 ± 395 | 1952 ± 302 | 1858 ± 515 |
| Amniotic fluid | | | | | |
| characteristics | | | | | |
| WBC (cells/mm³: | 10 [0-80] | 1800 [335-19200] | 520 [200-14800] | 23 [3-90] | 3 [0-62] |
| median [range]) | | | | | |
| Positive Gram stain | 0 | 13 | 0 | 3 | 1 |
| Histologic | 1 | 19 | 7 | 3 | 5 |
| chorioamnionitis | | | | | |
| and/or funisitis | | | | | |

GA: gestational age; WBC: white blood cell count (+WBC: WBC count >100 cells/mm3); AFC: Amniotic fluid cultures; (+AFC: positive amniotic fluid culture results). The “diseased” and “non-diseased” groups are the two extreme groups used for learning SELDI profiles.

Diluted amniotic fluid from each subject was assigned to duplicate chips, and on each chip two spots were covered with 2 μl of PBS alone. The matrix consisted of either 1 μl of a 20% saturated solution of α-cyano-4-hydroxycinnamic acid (CHCA) on one set of chips, or two sequential applications of 0.5 μl saturated solution of sinnapinic acid (SPA) on the other. The chips were air-dried and were then read in a Protein Biology System® II (PBS II) SELDI-TOF mass spectrometer (Ciphergen Biosystems), using the ProteinChip® software, versions 2.lb and 3. The chip covered with CHCA was analyzed first using a low-laser intensity spot protocol (CHCA-LL: laser intensity 220, mass ranging from 0 to 20,000 Da, optimized between 1000 and 10,000 with detector sensitivity 6, mass focus 3300 Da, 20 shots fired and averaged for every 5-th position from starting from position 20 to 80), followed by a high-laser intensity spot protocol (CHCA-HL: laser intensity 240, detector sensitivity 10, 25 shots fired and averaged for every 5-th position from position 22 to 82). The chip covered with SPA was analyzed with a single spot protocol (SPA: laser intensity 285, mass ranging from 0 to 200,000 Da, optimized between 20,000-90,000 Da with mass focus at 26,500 Da, detector sensitivity 10, and 20 shots fired and averaged for every 5-th position from position 20 to 80). The PBS II instrument was calibrated externally against four molecular weight peptide standards: arg-8-vasopressin, bovine insulin β chain, human insulin, and hirudin.

### Procedures for Biomarker Identification and Quantification

Isoelectric points (pH where the protein has no net charge) for the peaks of interest were estimated by spotting diluted amniotic fluid onto WCX2 and SAX2 chips and washing individual spots with solutions of incremental pH (50 mM sodium acetate pH=4, 50 mM sodium phosphate pH=6, 50 mM Tris pH=8 or 50 mM sodium carbonate pH=10) before SELDI analysis. If the pH of the buffer solution is below its isoelectric point, a protein will bear a net positive charge binding preferentially to the WCX2. If the pH of the buffer solution is above its isoelectric point, it will bind preferentially to the SAX2 surface. Thus, the isoelectric point of a protein of interest would be between the pH of the buffer solutions where a peak appears on the SAX2 and the WCX2 chip, respectively.

In-gel trypsin digest. ID-SDS-PAGE electrophoresis was carried out on precast 10-20% tris-tricine gels (5-well gels, InVitrogen, Carlsbad, Calif.). Samples were prepared by boiling amniotic fluid for 5min. with an equal volume of Tricine loading buffer (BioRad, Lajolla, Calif.) under non-reducing conditions. The gels were loaded with 20 μl sample volume per well, run at 120V, stained with 0.1% Comassie Blue R250 in 40% methanol/10% acetic acid for 1 hour and then destained with repeated changes of 40% methanol/10% acetic acid. Low molecular weight markers (Ultralow Color marker, product of Sigma, St. Louis, Mo.) or a mixture of mass spectrometry molecular weight standards (0.5 nmols of bovine cytochrome C and 0.5 nmols of bovine ubiquitin, Ciphergen Biosystems) were loaded on gels along with the amniotic fluid samples.

Bands of interest were precisely cut out, using a scalpel blade, and were minced, and in-gel tryptic digestion then was performed using a peptide mapping kit, a product of Ciphergen Biosystems (Fremont, Calif.). At the end of the procedure, 50 μl of proteomics sequencing grade trypsin (Sigma) containing 0.2 μg enzyme in 0.4 mM HCl/25 mM ammonium bicarbonate was added to each tube containing the gel pieces and incubated for about 16 hours in an oven at about 37° C. A piece of blank gel was processed along with the protein bands to differentiate between products resulting from trypsin autolysis and fragments from the protein of interest. Peptide maps were read manually in the PBS II instrument, after 1.5 μl of each digest was dried directly onto H4 spots and then overlaid with 1 μl 10% saturated CHCA solution.

Peptide elution. A few gel pieces from each band were placed in a sonic bath in 20 μl of 50% formic acid/25% acetonitrile/15% isopropanol for 30 minutes. The solution then was removed and evaporated under vacuum, and the eluted proteins resuspended in 10 μl water, of which 5 μl were dried down onto H4 spots, overlaid with two applications of 0.5 μl saturated SPA solution and read manually in the PBS II instrument.

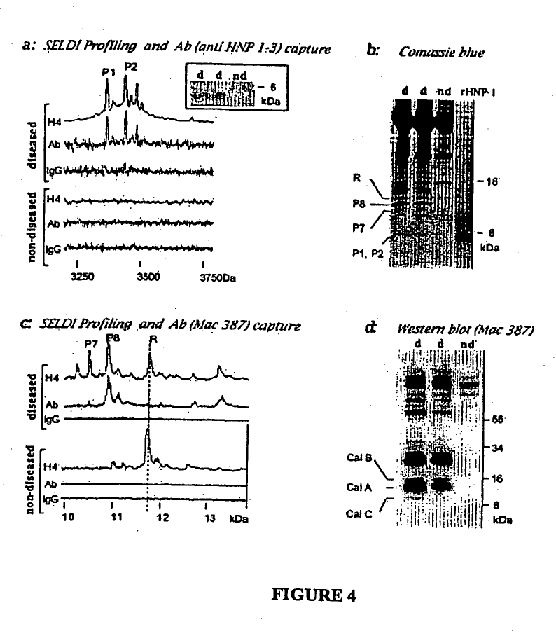

Gel staining and western blotting. 5 μl of amniotic fluid diluted 1:1 with tricine sample buffer (Bio-Rad) were boiled for 5 min. and loaded under onto 16% continuous (for HNP-1-3; see FIG. 4b) or 10-20% gradient (for calgranulins; see FIG. 4c) precast tricine, product of InVitrogen Corporation (Carlsbad, Calif.). Gels either were stained with Comassie blue (FIG. 4b) or were transferred electrophoretically to PVDF membrane filters for western blotting (FIG. 4c). Briefly, filters were blocked with 5% milk and then incubated with either mouse Mac 387 monoclonal antibody for calgranulins (1:1000 dilution; Labvision, Fremont, Calif.) or rabbit polyclonal anti HNP-1-3 (1:1000 dilution, rabbit anti-human HNP-1-3; Abcam, Cambridge, UK) for 1 hour (h) at 25° C. Detection was performed using appropriate horseradish peroxidase-linked secondary antibody and ECL-kit (Amersham Biosystems).

On-chip immunoassay. Spots on PS20 arrays (epoxy-activated for covalent immobilization of proteins) were incubated for 1 h with 2 μl affinity purified antibody solution (Mac 387 for calgranulin capture or anti human HNP-1-3) at 1 mg/ml diluted in PBS as purchased from the manufacturer. Paired chips were spotted with mouse or rabbit 1 gG, as appropriate, in a similar concentration. Unreacted active sites were blocked by incubation with 4 μl per spot 1M Tris, pH=9 for 20 min. at room temperature. The chips were washed with PBS supplemented with up to 850 mM NaCl and 0.05% Tween-20 (binding buffer). A 5μl sample (diluted progressively in binding buffer from 1:10 to 1:80 for calgranulin or 1:500 to 1:64,000 for HNP capture) was incubated on the pre-treated spots. After about one hour, the spots were vigorously washed, first with binding buffer and then with 10 mM HEPES, air-dried, covered with appropriate matrix solution, and read in the PBS II system.

ELISA. Concentrations of HNP-1-3 in amniotic fluid were measured with a commercially available enzyme linked immunosorbent assay (HyCult Biotechnologies, Uden, The Netherlands) with a sensitivity of 19.5 pg/mL. Intra-assay and inter-assay coefficients of variation were <2%.

### Statistical Analysis

Data were tested for normality using the Kolmogorov-Smirnov test and compared with Mann-Whitney test (nonparametric), Student's t test (parametric), or one-way ANOVA (parametric for comparisons among multiple groups). Comparisons between proportions were performed using the Fisher's exact test. Receiver operating characteristic (ROC) curve analysis, inter-rate agreement and kappa calculations were performed using MedCalc statistical software (MedCalc, Broekstraat, Belgium).

### Results

(1) “Learning” SELDI Protein Profiles in Amniotic Fluid

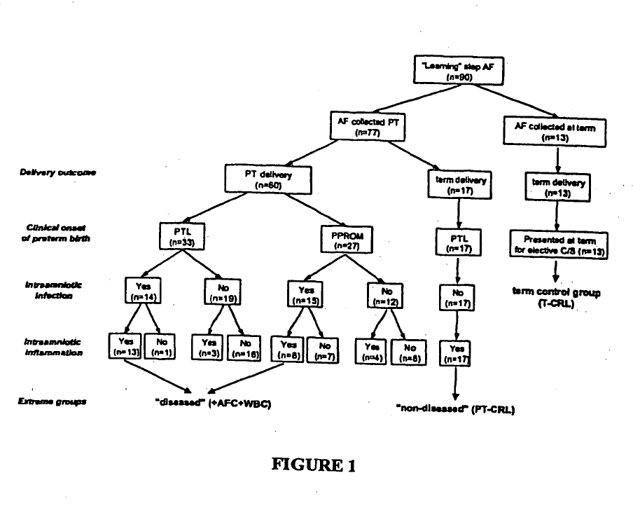

Amniotic fluid samples from 90 pregnant women were analyzed (FIG. 1). Seventy-seven samples were obtained from subjects with the diagnosis of preterm labor (n=50) or preterm PROM (n=27) (average gestational age: 29 weeks [95% Cl: 28-30]) and 13 from subjects at term, not in labor (average gestational age: 39 weeks [95% Cl: 39-40]). The first step was to generate two groups with extreme clinical and biological characteristics, namely, a “diseased” and a “non-diseased” group. Subjects who delivered a preterm neonate and had evidence of infection and inflammation were selected to represent the “diseased” group. Infection was defined as a positive amniotic fluid culture for microorganisms, whereas intra-amniotic inflammation was defined as an amniotic fluid WBC count>100 cells/mm³. The “non-diseased” group was constituted of subjects with premature labor with intact membranes who subsequently delivered a term neonate without complications (preterm control group: PT-CRL). Subjects at term at the time of amniotic fluid retrieval represented an additional control group (term control group: T-CRL). The distribution of subjects according to clinical presentation (preterm parturition or term gestation), preterm delivery (<37 weeks), amniotic fluid cultures and amniotic fluid WBC count is illustrated in FIG. 1. The clinical characteristics of the subgroups are displayed in Table 1. The extreme “diseased” and “non-diseased” groups are in the first two columns.

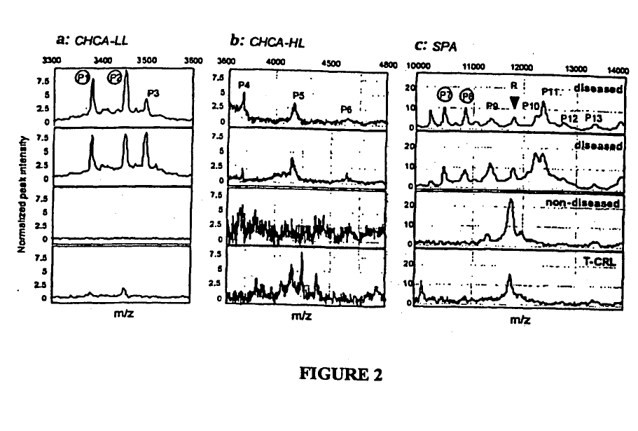

Preliminary data mining consisted of visual inspection of the SELDI protein profile tracings followed by evaluation with the “biomarker wizard tool” at a 0.3% mass accuracy and biomarker statistics. This analysis suggested that informative peaks were clustered within three m/z (ratio mass/charge) areas. These areas of interest were between 3300-3600 Da in the CHCA-LL spectra, 3600-5000 Da in the CHCA-HL spectra and 10,000-14,000 Da with SPA (FIG. 2). Conspicuous peaks in these zones were selected manually and the spectra from each subject verified for accuracy of peak identification. The m/z value, normalized intensity and signal to noise ratio (S/N) for the selected peaks were extracted. Next, a stepwise strategy was established to define rigorous filter preferences for our diagnostic proteomic profile. The criteria included the following: (1) all peaks should be present in the “diseased” state. Thus, the search was for new peaks in “diseased” subjects, rather than disappearance or decrease of peaks normally present in “non-diseased” subjects; (2) the peaks of the profile should have been detected on at least two different laser intensities or matrix protocols; (3) all peaks in the profile should be significantly different (in logarithm of normalized intensity) at least at a level of p<0.0001 between the “diseased” and “non-diseased” groups; (4) only parent peaks were considered (singly ionized, least oxidized); (5) peaks should not have occurred in areas where the baseline noise in “non-diseased” individuals is significantly elevated; and (6) the final diagnostic profile should be parsimonious.

After applying the first four criteria, 13 candidate peaks with potential discriminatory value emerged (FIG. 2). To objectively score the peaks as present or absent, the evaluation of the S/N ratio was undertaken. The cut-off used for selection was the mean ±2 standard deviations of the S/N ratio for each corresponding mass in the “non-diseased” group. Boolean indicators were then assigned: a value of 0 was used if a peak was absent or below the cut-off, and a value of 1 was assigned for peaks above the cut-off. The sum of Boolean indicators was computed for each subject and is referred as the M score (Mass score). This score represents the sum of observations in the three different matrix protocols (for example, the first two subjects in FIG. 2 have M scores of 12, while the third and the fourth have scores of 0 and 3, respectively).

To select the peaks not occurring in areas of baseline noise in “non-diseased” individuals (fifth criterion), the noise level for each matrix protocol (CHCA-LL, CHCA-HL and SPA) was analyzed at the 13 corresponding average masses on the spectra from the intermingled spots with PBS instead of amniotic fluid (n-20). Peaks where the average noise level (S/N at the corresponding m/z value) in “non-diseased” group was significantly higher (p<0.05) than the noise level with PBS alone were eliminated. This criterion was chosen to minimize overlap of informative peaks with those present nearby in the tracing of normal women. Another advantage of this approach was that objective quantitative and qualitative analysis of the peaks of interest was now possible and independent of the availability of “non-diseased” samples, which would be a substantial obstacle in the clinical implementation of the test.

After sequentially applying the filtering strategy outlined above, a set of four peaks was obtained, which defined the final proteomic profile. The sum of 0 or 1 values for each of these four peaks computed the MR score (Mass restricted score), ranging from 0 to 4 (the peaks selected for calculation of the MR are displayed in FIG. 2 within the circle; the first two subjects have MR scores of 4, the third and the fourth of 0 and 2, respectively). Therefore, each subject had two scores: the M score and the MR score. In Table II, the peaks composing the M and the MR score are listed with their observed mass, as derived only from the peaks with a Boolean indicator of 1 (i.e., peak is present).

In addition to the 13 initial peaks composing the M proteomic profile, a distinct peak of approximately 11.7 kDa (defined as reference or R peak, FIG. 2) was present in all fluids. This peak is useful for the quick visual orientation to the m/z axis of the SPA spectra.

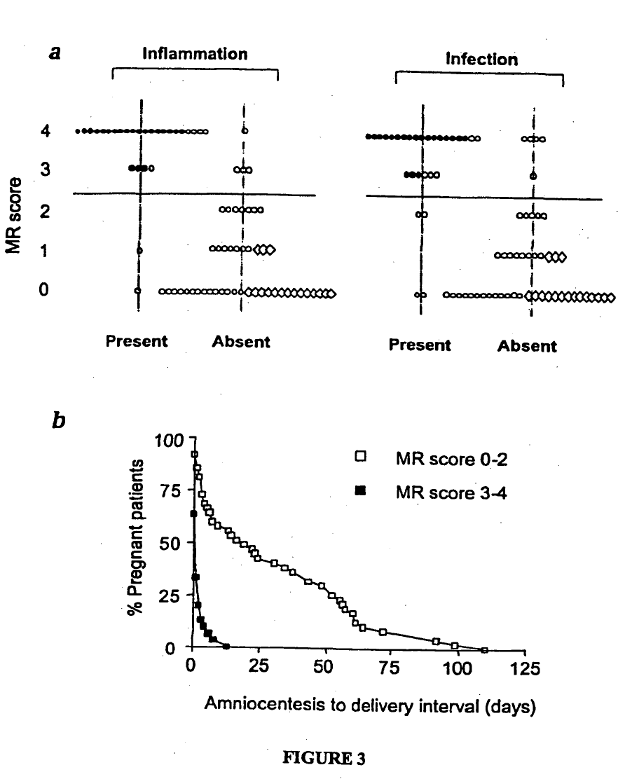

To test the diagnostic performance of the M and MR scores in the discrimination of “diseased” and “non-diseased” states, receiver operating characteristic curve analysis was performed. The M and MR scores performed essentially identically in delineating between subjects with infection and inflammation and those with premature labor who delivered at term. This indicates that the 4 peaks composing the MR score are sufficient for the stated purposes (i.e., parsimony). All subjects with preterm labor who delivered at term had an MR score of either 0 or 1 (open diamonds in FIG. 3a). All subjects with inflammation and infection (closed circles in FIG. 3a) had an MR score of 3 or 4. Thus, an MR score >2 had 100% sensitivity and 100% specificity in the discrimination of these two extreme clinical conditions.

To determine whether the MR score could discriminate between preterm subjects with intra-amniotic inflammation and those with microbiologically proven infection, the samples from the entire population were evaluated. An MR score over 2 had a sensitivity of 92.9% (26/28) and a specificity of 91.8% (45/49) in the detection of intrauterine inflammation (area under the ROC curve=0.948, SE=0.031, 95% Cl: 0.871 to 0.985). Two subjects had WBC>100/mm³ with low MR scores (false negative for inflammation) (FIG. 3a). Both had negative microbial cultures. One presenting with preterm PROM at 28 weeks of gestation had 200 WBC/mm³ and delivered prematurely after 9 days. The other subject presenting 27 weeks of gestation had 375 WBC/mm³ and delivered at 30 weeks of gestation during a second admission for abdominal trauma. There were 4 subjects with WBC<100/mm³ who had MR scores of 3 or 4 (false positive for inflammation). All presented with preterm PROM at 30-33 weeks of gestation, delivered within 3 days, and in all Ureaplasma urealyticum or Mycoplasma hominis were isolated from the amniotic fluid.

An MR score greater than 2 identified intra-amniotic infection with 86.2% sensitivity (25/29), 89.6% specificity (43/48) (area under the ROC curve=0.893, SE=0.042, 95% Cl: 0.802 to 0.952) (FIG. 3a). Four subjects had positive amniotic fluid cultures for microorganisms but did not display the characteristic profile (i.e., false negative for infection: 2 subjects had a score of 0 and 2 an MR score of 2 due to P1 and P2 presence). The two subjects with an MR of 0 had amniotic fluid WBC counts below 5 WBC/mm³ (no intra-amniotic inflammation). One subject was admitted at 33 weeks of gestation with preterm PROM and delivered within two days after labor induction. The amniotic fluid culture was positive for Streptococcus agalactiae, the neonate weighed 2160 grams, had Apgar scores of 9/9 and no complications. The second subject was admitted at 29 weeks of gestation with preterm labour and intact membranes, had a positive amniotic fluid culture for Streptococcus viridans, and delivered 34 days later (at 34 weeks of gestation). The neonate weighed 2260 grams and had no complications. Neither one of these two subjects had histological evidence of chorioamnionitis. There were two subjects with MR scores of 2 also representing apparent false negative results. Both presented with pre term PROM and amniotic fluid cultures were positive for Prevotella oralis or Gardenella vaginalis, but without evidence of intra-amniotic inflammation. Both subjects were induced and delivered within 5 days, but the placentas had no evidence of acute chorioamnionitis at delivery. Thus, the apparent false negative cases of the proteomic analysis did not have any clinical or histological evidence of inflammation or complications of prematurity.

There were 5 subjects with abnormal MR scores (3-4) and negative amniotic fluid cultures (false positive results for infection). All had amniotic fluid WBC counts over 100/mm³, evidence of histological chorioamnionitis and delivered preterm neonates shortly after admission. Thus, all apparent false positive results were associated with abnormal outcomes and histological evidence of acute inflammation.

Survival analysis of the duration of pregnancy demonstrated that subjects with an MR score of 3 or 4 had a shorter amniocentesis to delivery interval than those with an MR score of 0 to 2 (log rank Mantel-Haenszel test Chi square 42.6, p<0.0001). The median amniocentesis to delivery interval was 1 day in subjects with MR scores of 3 or 4, but 19 days for subjects with MR scores of 0, 1 or 2 (95% Cl of ratio: 18-20 days) (FIG. 3b). The difference remained highly significant when 26 subjects delivered for maternal or foetal indications were censored (median amniocentesis to delivery interval for subjects with MR scores of 0-2=43 days vs. 2 days for MR scores of 3-4; log rank Chi-square 22.7, p<0.0001, ratio: 21.5 days, 95% Cl of ratio: 21-22).

(2) Test of the MR Score in a Different Population of Preterm Subjects

The performance of the MR score was examined by analyzing blindly a separate set of 24 samples of amniotic fluid. Two investigators selected from the fluid bank 12 “diseased” and 12 “non-diseased” samples. The selection criteria for the diseased subjects were: 1) diagnosis of preterm labor with intact membranes; 2) intra-amniotic inflammation (amniotic fluid WBC>100/mm³) and acute histological chorioamnionitis; 3) spontaneous preterm delivery shortly after amniocentesis. The latter group consisted of two subgroups of subjects: 6 with a positive amniotic fluid culture for microorganisms and 6 with a negative amniotic fluid culture. Control subjects were those with the diagnosis of preterm labor and intact membranes, before 33 weeks, that had no elevated WBC in amniotic fluid and negative cultures, and delivered at term. The selection criteria were chosen to test the profile in subjects with either clearly defined disease or absence of disease as in premature labor and in contrast to other conditions (e.g., cancer) no real “gold standard” for disease is available to classify subjects at the time of amniotic fluid analysis. Samples were randomly coded and two other investigators performed the SELDI experiments, as described, and independently analyzed and scored the spectra. The MR scores of 3 or 4 diagnosed with 100% sensitivity and specificity the presence of intra-amniotic inflammation. In addition, by quick visual evaluation of the presence or absence of the peaks composing the MR score on the CHCA-LL and SPA SELDI protocols relative to the R peak present in all samples, the investigators were able to subjectively diagnose inflammation again with 100% sensitivity and specificity. There was 100% inter-rater agreement (kappa=1) on scores and diagnosis. Thus, this example identifies intra-amniotic inflammation.

(3) Identity of Biomarkers Composing the MR Score

To determine that P1 and P2 correspond to neutrophil defensins, an on-chip immunoassay was performed, using a polyclonal antibody, that does not distinguish between the three HNP peptides as each differ just by one amino acid.

FIG. 4a illustrates that the P1, P2 and P3 peaks present in profiling spectra (on H4 spots) also are detected on the spots pre-coated with the anti-HNP-1-3 antibody but not the 1 gG coated spots. In addition, the presence of the peptides was confirmed in samples from “diseased” subjects at the appropriate mass on Comassie stained gels (FIG. 4b) and by western blotting (FIG. 4a—insert).

The accuracy of mass determination was estimated by comparing the average masses of P1 and P2 in amniotic fluid with the average masses of the peaks obtained by spotting recombinant HNP-1 and HNP-2 peptides on triplicate spots of a mixture of equal amounts (0.5 ng each). The observed (in amniotic fluid) and calculated (SwissProt) masses for HNP-1 and 2 are listed in Table 2. The intra-assay variability calculated from the triplicate spots with recombinant peptides was 0.07% and the mass accuracy in both simple (mixture of recombinant peptides) and complex (amniotic fluid) samples was 0.2%.

TABLE 2
Biomarkers composing the M and MR scores

| Biomarker | Observed mass [95% Cl]; P1 | Calculated mass; P1 | Protein ID |
|---|---|---|---|
| P1 | 3378.2 [3377.0-3379.4]; P1 > 8 | 3377.01; 8.67 | HNP-2 |
| P2 | 3449.7 [3448.5-3451.0]; P1 > 8 | 3448.09; 8.68 | HNP-1 |
| P3 | 3493.0 [3491.4-3494.5] | | |
| P4 | 3720.7 [3719.5-3721.9] | | |
| P5 | 4149.0 [4147.5-4050.4] | | |
| P6 | 4629.6 [4603.5-4655.7] | | |
| P7 | 10471.7 [10467.9-10475.5]; P1: 4-6 | 10443.85; 5.82 | Calgranulin C |
| P8 | 10874.4 [10868.6-10880.2]; P1: 4-6 | 10834.51; 6.51 | Calgranulin A |
| P9 | 11362.3 [11355.2-11369.3] | | |
| R peak | 11790.4 [11785.9-11794.9] | | |
| P10 | 12221.0 [12213.8-12228.1] | | |
| P11 | 12354.4 [12345.3-12363.5] | | |
| P12 | 12730.5 [12724.5-12736.4] | | |
| P13 | 13329.2 [13323.2-13335.3] | | |

Circled peaks are components of the MR score;
PI: isoelectric point;
R peak: reference peak

To establish the identity of P7 and P8, first in-gel tryptic digest was performed as described above. Concurrently, the exact mass of the band to be digested was determined, by eluting the intact protein from pieces of the excised gel and subjecting it to mass spectrometry. This technique confirmed that an excised gel band corresponded indeed to the peak of interest on SELDI.

The observed mass of the intact protein, isoelectric point (Table II), and tryptic peptide masses were entered in searchable databases:

ProFound (http://129.85.19.192/profound_bin/WebProFound.exe),

Peptldent (http:www.expasy.ch/tools/peptident.html), and

MS-Fit (http://prospector.ucsf.edu/ucsfhtm14.0/msfit.htm). P7 was matched to calgranulin C (accession #P80511) and P8 to calgranulin A (accession #P05109), after which an on-chip immunoassay was performed, using a monoclonal antibody that recognized calgranulins A, B and C (1:1000, Mac 387) (FIG. 4c). FIGS. 4b and 4d show the bands of interest on a Comassie stained gel and conventional western blotting, using the same antibody used for the on-chip immunoassay.

It was then determined whether a rapid SELDI analysis of amniotic fluid, pursuant to the invention, also could provide quantitative information about the degree of intra-amniotic inflammation, a significant prognostic indicator of fetal outcome.

This information has been unavailable to practitioners, except for the WBC count of amniotic fluid and clinical symptoms (maternal fever, uterine tenderness or fetal tachycardia) as the rest of the rapid tests (e.g., Gram stain) diagnose infection rather than inflammation.

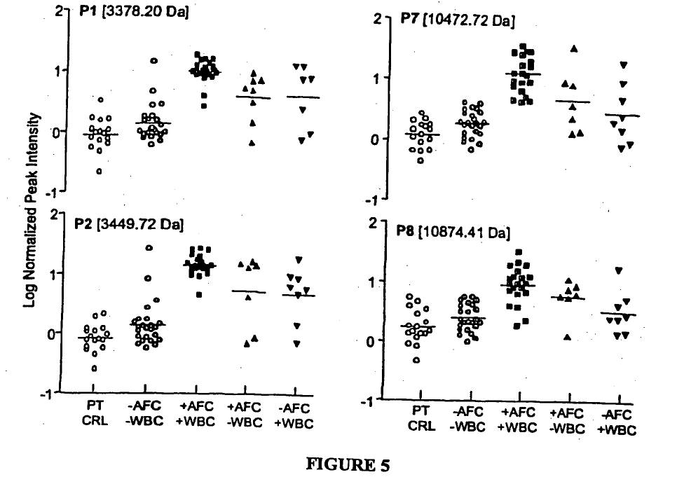

For this purpose, the log-normalized intensity of the MR peaks was analyzed from the 77 preterm subjects studied in the “learning” phase. After logarithmic transformation, all data sets of peak intensities were normally distributed. There was a significant difference in the mean signal intensity of all 4 peaks between the extreme “non-diseased” and “diseased” groups (p<0.001) (FIG. 5). There was also a significant difference in peak intensities between subjects with preterm labor who delivered at term (PT-CRL) and the group without infection or elevated WBC count that delivered preterm (-AFC-WBC, in Table I and FIG. 5), suggesting that at least in some of the subjects delivering preterm, there is evidence of intra-amniotic inflammation even with 20 WBC counts<100/mm³.

(4) SELDI Profile of Amniotic Fluid at Term

Five of the 13 samples of amniotic fluid at term had detectable P1 and P2 peaks on SELDI (i.e., above the S/N cut-off) (FIG. 2a). However, none showed visible P7 or P8 peaks (FIG. 2c), and thus were diagnosed as “non-diseased” by MR. This observation is consistent with increased availability of HNPs in amniotic fluid with advancing gestational age. In addition, it suggests dissociation between the presence of neutrophil defensins (HNP-1 and -2) and calgranulins in amniotic fluid, while emphasizing the value of MR score as a composite diagnostic test.

HNP-1-3 are peptides with antimicrobial activity involved in innate immunity and are present in high amounts in azurophilic granules of activated neutrophils. Accordingly, the intensity of the HNP peaks (P1 and P2) reflects the degree of neutrophil activation in the amniotic cavity, and since these cells are thought to be of fetal origin, the extent of fetal inflammation.

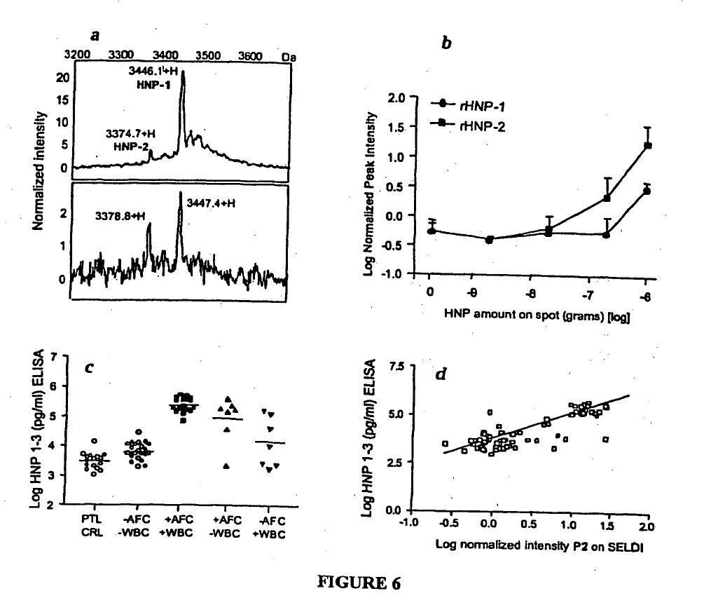

Between 20 ng and 1 μg (peptide amount on-chip) there was a dose-dependent relationship with the normalized peak intensity (FIGS. 6a and 6b). The limit of detection for HNP-1 was 5.8 fmols. Interestingly, in the presence of HNP-1, the intensity of the HNP-2 peak was significantly lower, suggesting either a peak suppression effect or differences in ionizing abilities in the mass spectrometer between the two peptides (FIG. 6a). These results identified HNP-1 (P2) as a better quantitative indicator of intra-amniotic inflammation than HNP-2 (P1).

To examine the accuracy of quantitative estimation in a complex sample such as amniotic fluid, the concentration of peptides by ELISA was also measured. This assay quantifies the total HNP-1-3 amount since the antibody used recognizes a common epitope on the three peptides. ELISA confirmed the significant differences in HNP 1-3 concentrations among groups seen with SELDI (FIG. 6c). There was a significantly linear correlation between the log of the HNP amount measured by ELISA and that of the normalized peak intensity (r²=0.701 for P1, 0.703 for P2) (FIG. 6d). The correlation between HNP-1-3 estimated by SELDI and amniotic fluid white blood cell count (log WBC/mm³) was weaker (r²=0.491 for P1, 0.501 for P2) than that observed between SELDI and HNP-1-3 by ELISA.

### EXAMPLE 2

This example illustrates that detection of the disclosed biomarkers calprotectin, BPI, and defensin in a sample of amniotic fluid from a subject indicates a diagnosis of preterm complications, such as preterm parturition and intra-amniotic inflammation.

In this example, samples of amniotic fluid from women in four groups were analyzed. Group 1 consisted of women in the mid-trimester (14-18 weeks) of pregnancy who underwent amniocentesis for genetic indications and delivered normal infants at term (n=84). Group 2 included women with preterm labor and intact membranes who were subdivided into the following categories: those with preterm labor who delivered at term with a negative amniotic fluid culture for micro-organisms (n=36); those with preterm labor who delivered preterm (<37 weeks) with a negative amniotic fluid culture for micro-organisms (n=52); and those with preterm delivery with MIAC (n=26). Preterm labor was defined by the presence of regular uterine contractions occurring at a frequency of at least two every 10 min and cervical changes before 37 completed weeks of gestation. MIAC was defined as a positive amniotic fluid culture for micro-organisms. Group 3 consisted of women with preterm PROM with (n=26) and without (n=26) MIAC. PROM was diagnosed as amniorrhexis before the onset of spontaneous labor. Membrane rupture was diagnosed with the use of vaginal pooling, by feming, or by a positive nitrazine test. The indications for amniocentesis in subjects of both groups 2 and 3 were for the detection of MIAC and fetal lung maturity. Group 4 included women with term gestations (37 weeks of gestation) with intact membranes and without MIAC. This group was subdivided into two subgroups composed of those not in labor (n=31) and those in labor (n=52). Women at term underwent amniocentesis for the assessment of lung maturity prior to Cesarean section or for the diagnosis of MIAC. Amniotic fluid not required for clinical purposes was centrifuged at 4° C. for 10 min. to remove cellular and particulate matter, and stored at −70° C. A sample of amniotic fluid was transported to the laboratory for culture of aerobic/anaerobic bacteria and Mycoplasma species. Amniotic fluid white blood cell (WBC) counts and assessment of glucose concentrations were not performed in some cases. The results of these tests were used for subsequent clinical management.

Concentrations of BPI, HNP 1-3 and MRP8/14 in amniotic fluid from the subjects in this example were determined with commercially available enzyme-linked immunoassays (Table 3).

TABLE 3
Table Sensitivity, interassay and intra-assay coefficients of variation (CV) of study assay kits

| | | | | Intra-assay CV | Interassay CV |
|---|---|---|---|---|---|
| Analyte | Company | City, country | Sensitivity | (%) | (%) |
| BPI | HyCult Biotechnology | Uden, The Netherlands | 71.25 pg/ml | 1.74 | 4.0 |
| HNP 1-3 | HyCult Biotechnology | Uden, The Netherlands | 19.46 pg/ml | 1.97 | 1.24 |
| Calprotectin (MRP 8/14) | BMA Biomedical AG | Augst, Switzerland | 0.686 ng/ml | 7.3 | 11.7 |

BPI: bactericidal/permeability-increasing protein; HNP 1-3: human neutrophil defensins 1, 2 and 3

The assays are specific for human proteins and were validated for amniotic fluid prior to use. Briefly BPI, HNP 1-3 and calprotectin immunoassays were based on the sandwich format. During the first step of the assays, amniotic fluid specimens were incubated in duplicate where the analyte present in the samples or the standard binds to the microtiter plate precoated with specific antibodies. Following incubation, repeated washings were performed to remove unbound materials. Step two involved incubation with biotinylated second antibody, which binds to the captured antigen in the microtiter plate to form a sandwich. After removing the excess and unbound materials, streptavidin peroxidase conjugate was added. The detection of bound peroxidase in each well was determined by the addition of tetramethylbenzidine (TMB) substrate in each of these assays. The reaction was stopped by the addition of either 2M citric acid of 1M sulfuric acid, and the resultant color was measured at 450 nm in an automated microtiter plate-based spectrophotometer (Bio-Tek Instruments, Winooski, Vt., USA.). The concentrations of individual analytes in amniotic fluid were determined by interpolation from the respective standard curve. The calculated sensitivity, inter- and intra-assay coefficients of variation for BPI, HNP 1-3 and calprotectin immunoassays are displayed in Table 3.

### Placental Pathology

The presence or absence of acute inflammatory lesions in the extraplacental membranes (histologic chorioamnionitis) and the umbilical cord (funisitis) was assessed as in subjects with preterm labor with intact membranes, as well as those with preterm PROM who delivered within 72 hours (h) of amniocentesis. This period of time was selected to preserve a meaningful temporal relationship between antimicrobial peptide amniotic fluid concentrations and placental pathologic findings.

### Statistical Analysis

The Shapiro-Wilk and Kolmogorov-Smirnov tests were used to test for normal distribution of the data. Since a normal distribution was not achieved after logarithmic transformation, Kruskal-Wallis and Mann-Whitney U tests were used to determine the differences of the median among groups. Spearman rank correlation was utilized to assess the correlations, and c2 or Fisher's exact test were used for comparisons of proportion variables. Among subjects with preterm labor and intact membranes, receiver-operating characteristic (ROC) curve analysis was employed for the identification of subjects who had MIAC, those who had intra-amniotic inflammation, and those who delivered within various intervals. The composite marker was recorded as a score of 1-4. Score 1 was given when none of the studied antimicrobial peptide concentrations was above the cut-off values and score 4 when all three markers were above the cut-off values. Cox regression analysis was applied to examine the interval from amniocentesis to delivery, according to antimicrobial peptide amniotic fluid concentrations, while controlling for other confounding factors. A p value of <0.05 was considered statistically significant (SPSS 10.0, SPSS Inc., Chicago, Ill. USA).

### Results

The results provided in this section demonstrate that a variety of preterm complications can be diagnosed (including prognosed) by detecting certain elevated levels of biomarkers. This section also provides examples of diagnostic (including prognostic) concentrations of the biomarkers.

Immunoreactive BPI was detectable in 61.6% (205/333) of the cases, immunoreactive calprotectin in 95.8% (318/332) and immunoreactive HNP 1-3 in all amniotic fluid samples. The gestational age at amniocentesis and the proportion of subjects with detectable BPI and calprotectin in each subgroup are displayed in Table 4. There were no significant differences in the median gestational age at amniocentesis among the three subgroups of subjects with preterm labor and intact membranes, or between the two subgroups of preterm PROM (p>0.05 for each). BPI was detected in only 9.5% (8/84) of mid-trimester samples from subjects with normal pregnancy outcome, whereas calprotectin was detectable in 84.5% (71/84) of the cases and HNP 1-3 in all amniotic fluid samples of this group.

TABLE 4
Table Gestational age at amniocentesis and the proportion of
samples that had detectable amniotic fluid antibacterial peptide
concentrations in each group

| | Gestational age | | | | | |
|---|---|---|---|---|---|---|
| | at amniocentesis | | BPI | | Calprotectin | |
| | (weeks) | | detectable | | detectable | |
| | Median | Range | % | n | % | n |
| Mid-trimester and normal outcomes | 16 | 14-18 | 9.5 | 8/84 | 84.5 | 71/84 |
| Preterm labor and delivery at term | 28.8 | 21.2-34.1 | 44.4 | 16/36 | 97.2 | 35/36 |
| Preterm labor and delivery preterm | 27.4 | 19.3-34.6 | 63.5 | 33/52 | 100 | 52/52 |
| Preterm labor with MIAC | 26.8 | 19-34.1 | 96.2 | 25/26 | 100 | 25/25 |
| Preterm PROM with no MIAC | 30.7 | 22-33.7 | 76.9 | 20/26 | 100 | 26/26 |
| Preterm PROM with MIAC | 29.1 | 19-33.4 | 96.2 | 25/26 | 100 | 26/26 |
| Term with no labor | 39.3 | 38-42 | 96.8 | 30/31 | 100 | 31/31 |
| Term and in labor | 39.3 | 37-41.5 | 92.3 | 48/52 | 100 | 52/52 |
| Total | 28.1 | 14-42 | 61.6 | 205/333 | 95.8 | 318/332 |

BPI, bactericidal/permeability-increasing protein; MIAC, microbial invasion of the amniotic cavity; PROM, premature rupture of membranes
Human neutrophil defensins 1-3 were detectable in all samples (333/333)

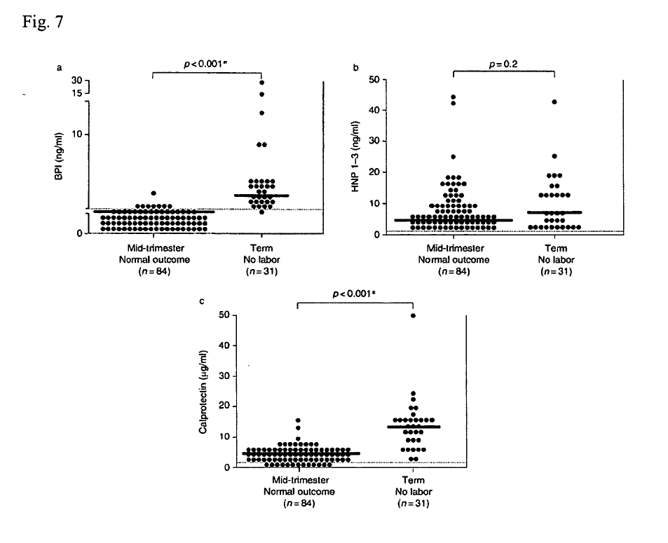

With reference to FIG. 7(a), the median amniotic fluid concentration of BPI in women at term without labor was significantly higher than in women in the mid-trimester (median 1.99 ng/ml, range 0-26.04 vs. median 0 ng/ml, range 0-2.41, respectively;p<0.001). FIG. 7(b) shows that the median amniotic fluid concentration of HNP 1-3 in women at term without labor was not significantly higher than in women in the mid-trimester (median 5.56 ng/ml, range 1.31-42.46 vs. median 2.8 ng/ml, range 1.21-43.89, respectively; p=0.2). FIG. 7(c) shows that the median amniotic fluid concentration of calprotectin in women at term without labor was significantly higher than in women in the mid-trimester (median 11.9 mg/ml, range 1.6-49.9 vs. median 1.45 mg/ml, range 0-14.4, respectively; p<0.001).

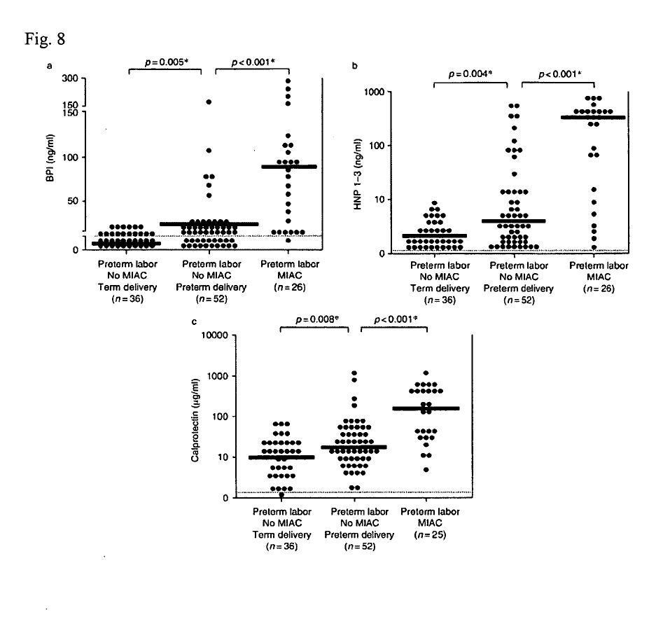

With reference to FIG. 8, (a) the median amniotic fluid concentration of BPI was significantly higher in women with preterm labor who delivered preterm than in women who delivered at term (median 0.88 ng/ml, range 0-169 vs. median 0 ng/ml, range 0-8.35, respectively; p=0.005). Similarly, the median amniotic fluid concentration of BPI in women with microbial invasion of the amniotic cavity (MIAC) was significantly higher than in women with preterm labor without MIAC who delivered preterm (median 71.59 ng/ml, range 0-285 vs. median 0.88 ng/ml, range 0-169, respectively; p<0.001). FIG. 8(b) shows that the median amniotic fluid concentration of HNP 1-3 was significantly higher in women with preterm labor who delivered preterm than in women who delivered at term (median 2.93 ng/ml, range 1.16-342.4 vs. median 1.71 ng/ml, range 1.13-9.28, respectively;p=0.004). Similarly, the median amniotic fluid concentration of HNP 1-3 in women with MIAC was significantly higher than in women with preterm labor without MIAC who delivered preterm (median 195.18 ng/ml, range 1.13-424.7 vs. median 2.93 ng/ml, range 1.16-342.4, respectively; p<0.001). FIG. 8(c) shows that the median amniotic fluid concentration of calprotectin was significantly higher in women with preterm labor who delivered preterm than in women who delivered at term (median 15.3 mg/ml, range 1.8-1023.3 vs. median 11.72 mg/ml, range 0-52.5, respectively; p=0.008). Similarly, the median amniotic fluid concentration of calprotectin in women with MIAC was significantly higher than in women with preterm labor without MIAC who delivered preterm (median 188.1 mg/ml, range 5.05-1013.9 vs. preterm delivery, median 15.3 mg/ml, range 1.8-1023.3, respectively; p<0.001).

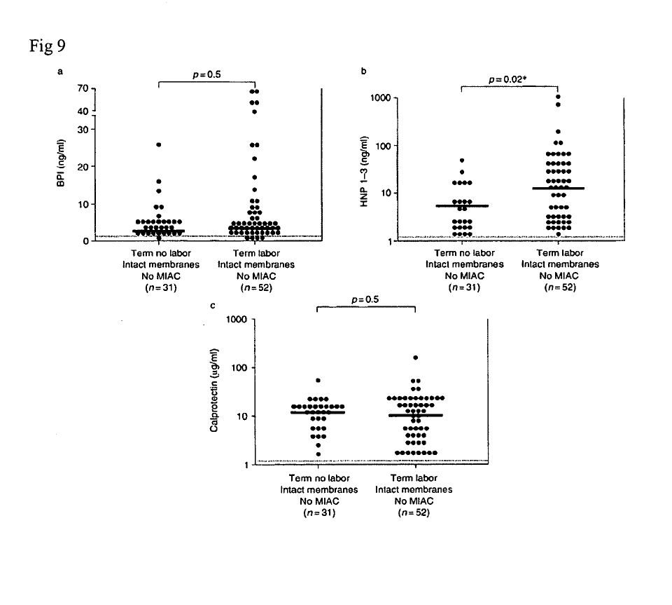

With reference to FIG. 9(a) the median amniotic fluid concentration of BPI was not significantly different in women at term with and without labor (median 2.59 ng/ml, range 0-68.21 vs. median 1.99 ng/ml, range 0-26.04, respectively; p=0.5). FIG. 9(b) shows that the median amniotic fluid concentration of HNP 1-3 was significantly higher in women at term in labor than in women at term without labor (median 12.1 ng/ml, range 1.14-982 vs. median 5.56 ng/ml, range 1.31-42.46, respectively; p=0.02). FIG. 9(c) shows that the median amniotic fluid concentration of calprotectin was not significantly different in women at term with and without labor (median 10.14 mg/ml, range 1.7-135.2 vs. median 11.9 mg/ml, range 1.6-49.9, respectively; p=0.5).

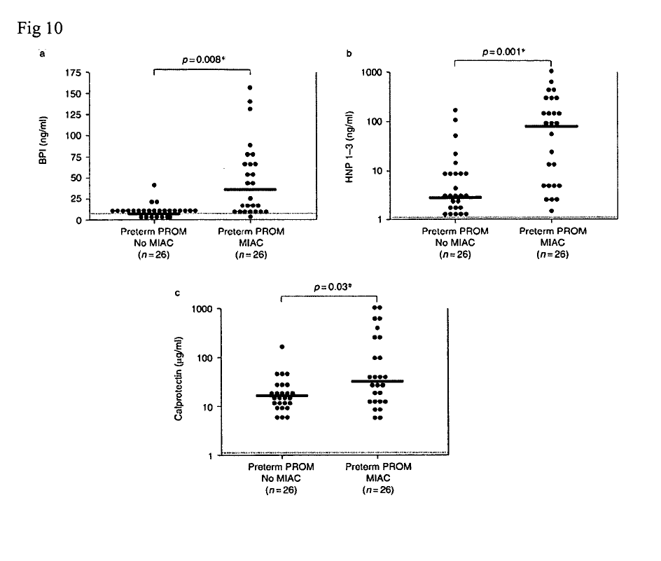

With reference to FIG. 10(a) the median amniotic fluid concentration of BPI was significantly higher in women with preterm PROM and with MIAC than in women with preterm PROM without MIAC (median 32.66 ng/ml, range 0-157 vs. median 2.03 ng/ml, range 0-35.26, respectively; p=0.008). FIG. 10(b) shows that the median amniotic fluid concentration of HNP 1-3 was significantly higher in women with preterm PROM and MIAC than in women with preterm PROM without MIAC (median 78.78 ng/ml, range 1.65-944.5 vs. median 2.92 ng/ml, range 1.23-157.65, respectively; p<0.001). FIG. 10(c) shows that the median amniotic fluid concentration of calprotectin was significantly higher in women with preterm PROM and MIAC than in women with preterm PROM without MIAC (median 29.13 mg/ml, range 5.4-906.1 vs. median 15.3 mg/ml, range 5.2-149.8, respectively;p=0.03).

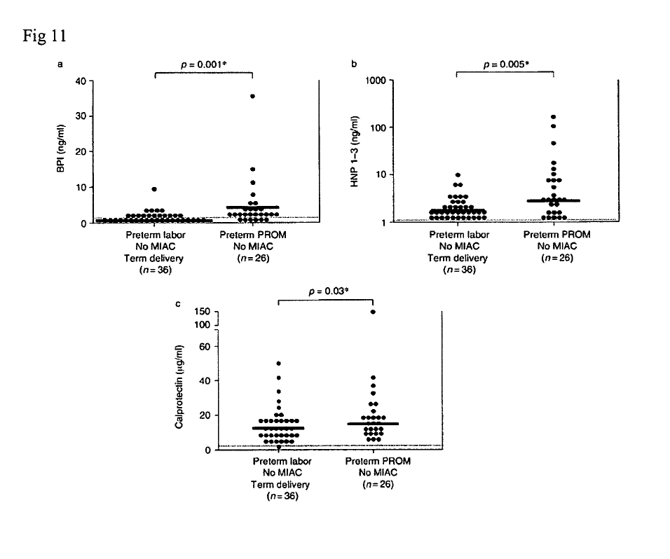

With reference to FIG. 11(a) the median amniotic fluid concentration of BPI was significantly higher in women with preterm PROM than in women with preterm labor and term delivery (median 2.03 ng/ml, range 0-35.26 vs. median 0 ng/ml, range 0-8.35, respectively; p=0.001). FIG. 11(b) shows that the median amniotic fluid concentration of HNP 1-3 was significantly higher in women with preterm PROM than in women with preterm labor and term delivery (median 2.93 ng/ml, range 1.23-157.65 vs. median 1.71 ng/ml, range 1.13-9.28, respectively;p=0.005). FIG. 11(c) shows that the median amniotic fluid concentration of calprotectin was significantly higher in women with preterm PROM than in women with preterm labor and term delivery (median: 15.3 mg/ml, range 5.2-149.8 vs. median 11.72 mg/ml, range 0-52.5, respectively;p=0.03).

As can be seen from FIG. 7, women at term and not in labor had significantly higher median amniotic fluid concentrations of BPI and calprotectin than women in the mid-trimester who delivered a normal neonate at term, but not of HNP 1-3. Furthermore, when the analysis includes women in the mid-trimester who delivered a normal neonate at term, women with preterm labor and intact membranes who delivered at term and women at term with intact membranes and not in labor, there was a positive correlation between amniotic fluid concentrations of both BPI and calprotectin with gestational age (Spearman's rho coefficient 0.7; p<0.001 for both). Hence BPI, calprotectin, or both, can be used as a marker of gestational age. In contrast, there was no significant correlation between the amniotic fluid concentration of immunoreactive HNP 1-3 and gestational age (Spearman's rho coefficient −0.002; p=0.9). As shown in FIG. 7, preterm parturition, in the absence of MIAC, was associated with a significantly higher median amniotic fluid concentration of HNP 1-3, BPI and calprotectin, whereas spontaneous labor at term was associated with a significantly higher median amniotic fluid concentration of only HNP 1-3, but not of BPI and calprotectin as can be seen in FIG. 8. As can be seen from FIGS. 7 and 9, MIAC was associated with a significantly higher median amniotic fluid concentration of HNP 1-3, BPI and calprotectin in women with preterm labor and intact membranes, and in those with preterm PROM than in women with sterile amniotic fluid (negative amniotic fluid culture). Similarly, FIG. 10 shows that in the absence of MIAC, women with preterm PROM had a significantly higher median amniotic fluid concentration of HNP 1-3, BPI and calprotectin than those with preterm labor and intact membranes who delivered at term. As can be seen in Table 5, there were positive correlations among the amniotic fluid concentrations of the three antimicrobial peptides and the amniotic fluid WBC count, and negative correlations among the concentrations of these antimicrobial peptides and amniotic fluid glucose concentrations, with the exception of calprotectin. Similarly, there were positive correlations among the amniotic fluid concentrations of the three antimicrobial peptides, except between calprotectin and HNP 1-3, as also can be see in from Table 5.

TABLE 5
Table Spearman's rho correlations of antibacterial peptide
concentration, white blood cell count and glucose concentration
in amniotic fluid

| | BPI | HNP 1-3 | Calprotectin |
|---|---|---|---|
| | (ng/ml) | (ng/ml) | (μg/ml) |
| Amniotic fluid white blood | 0.5 | 0.5 | 0.3 |
| cell count (cells/mm³) | p < 0.001* | p < 0.001* | p = 0.04* |
| Amniotic fluid glucose | −0.4 | −0.6 | NS |
| (mg/dl) | p = 0.003* | p < 0.001* | |
| BPI (ng/ml) | 1 | 0.3 | 0.58 |
| | | p < 0.001* | p < 0.001* |
| HNP 1-3 (ng/ml) | | 1 | NS |
| Calprotectin (μg/ml) | | | 1 |

*Statistically significant, p < 0.05;
NS, statistically non-significant
BPI, bactericidal/permeability-increasingprotein;
HNP, human neutrophil defensins

With reference to Table 6, among subjects who delivered within 72 hours (h) of amniocentesis, placental pathology was available in 89% (33/37) of those with preterm labor with intact membranes, and in 96% (27/28) of those with preterm PROM. Subjects with evidence of inflammation in the extraplacental membranes (histologic chorioamnionitis) and/or umbilical cord (funisitis) had a significantly higher median amniotic fluid concentration of BPI, HNP 1-3 and calprotectin than those without inflammation. Hence any one of the three, a combination of any two, or all three together, may be used as a marker for chorioamnionitis or funisitis.

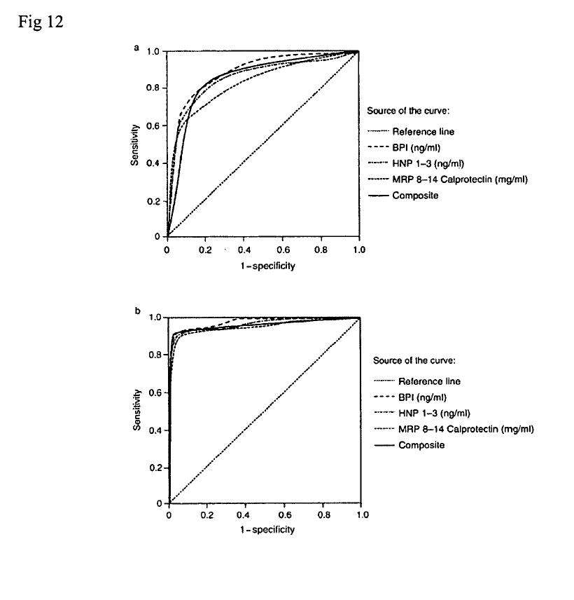

The diagnostic performance of each biomarker concentration in amniotic fluid was assessed among subjects who presented with preterm labor with intact membranes. Table 7 displays various exemplary diagnostic values (derived from ROC curves) of amniotic fluid concentrations of the three antimicrobial peptides for the identification of subjects who had MIAC, intra-amniotic inflammation (defined as amniotic fluid WBC count either ³ 50 or ³ 100 cells/ml), or those who delivered following spontaneous labor, within 48 h, 72 h and 7 days. The ROC curves for the identification of MIAC and intra-amniotic inflammation (defined as amniotic fluid WBC count ³ 50 cells/ml) are displayed in FIG. 12. The diagnostic efficacy of antimicrobial peptide concentrations and other previously published markers in amniotic fluid (Gram stain, WBC count≧50 cells/ml and glucose concentration≦14 mg/dl) for the identification of MIAC are displayed in Table 8.

TABLE 6
Table Amniotic fluid concentration of antibacterial peptides in pregnant women
with preterm labor and preterm premature rupture of membranes (PROM)
with and without placental inflammation

| | Preterm labor with intact membranes | | | Preterm PROM | | |
|---|---|---|---|---|---|---|
| | Without placental | With placental | | Without placental | With placental | |
| | inflammation | inflammation | p Value | inflammation | inflammation | p Value |
| | (n = 10) | (n = 23) | (a) | (n = 8) | (n = 19) | (b) |
| BPI (ng/ml) | 1.84 (0-22.11) | 7.52 (2.8-285) | <0.001* | 3.53 (0-13.76) | 52.65 (0.39-157) | 0.01* |
| HNP 1-3 (ng/ml) | 3.41 (1.3-89.55) | 201.4 (2.58-424.7) | <0.001* | 3.9 (1.25-18.72) | 81.7 (1.44-944.5) | 0.02* |
| Calprotectin (μg/ml) | 17.04 (7.17-120.9) | 212.72 (5.96-1013.9) | 0.002* | 12.15 (5.86-37.5) | 34.33 (5.44-90.61) | 0.02* |

BPI, bactericidal/permeability-increasingprotein; HNP, human neutrophil defensins
(a) Compared between pregnant women with preterm labor and intact membranes with and without placental inflammation
(b) Compared between pregnant women with preterm, PROM with and without placental inflammation
*Statistically significant, p < 0.05

TABLE 7
Table Diagnostic indices of each antibacterial peptide in amniotic fluid of patients
with preterm labor and intact membranes

| | | | Sensitivity | Specificity | Area | |
|---|---|---|---|---|---|---|
| Outcomes of interest | Prevalence | Cut-off value | (%) | (%) | (%) | p Value |
| MIAC | 22% (25/113) | | | | | |
| BPI (ng/ml) | | 3.7 | 80 | 79 | 87 | <0.001* |
| HNP 1-3 (ng/ml) | | 7.8 | 80 | 79 | 85 | <0.001* |
| Calprotectin (μg/ml) | | 27.9 | 80 | 78 | 84 | <0.001* |
| Composite markers | | any 2 or 3 markers ≧ cut-off | 80 | 83 | 84 | <0.001* |
| AF white blood cell count > 50 cells/ml | 21% (24/112) | | | | | |
| BPI (ng/ml) | | 15.5 | 92 | 96 | 96 | <0.001* |
| HNP 1-3 (ng/ml) | | 53.2 | 92 | 94 | 95 | <0.001* |
| Calprotectin (μg/ml) | | 48.4 | 92 | 93 | 94 | <0.001* |
| Composite markers | | all markers ≧ cut-off | 92 | 98 | 94 | <0.001* |
| AF white blood cell count > 100 cells/ml | 21% (23/112) | | | | | |
| BPI (ng/ml) | | 19.5 | 91 | 97 | 96 | <0.001* |
| HNP 1-3 (ng/ml) | | 62.1 | 91 | 94 | 95 | <0.001* |
| Calprotectin (μg/ml) | | 48.4 | 91 | 92 | 94 | <0.001* |
| Composite markers | | all markers ≧ cut-off | 91 | 98 | 94 | <0.001* |
| Spontaneous labor and delivery within 48 h | 25% (23/93) | | | | | |
| BPI (ng/ml) | | 2.7 | 83 | 81 | 89 | <0.001* |
| HNP 1-3 (ng/ml) | | 3.4 | 83 | 71 | 87 | <0.001* |
| Calprotectin (μg/ml) | | 18.5 | 78 | 73 | 82 | <0.001* |
| Composite markers | | any 2 or 3 markers ≧ cut-off | 83 | 82 | 85 | <0.001* |
| Spontaneous labor and delivery within 72 h | 29% (27/93) | | | | | |
| BPI (ng/ml) | | 1.5 | 85 | 80 | 88 | <0.001* |
| HNP 1-3 (ng/ml) | | 2.8 | 85 | 68 | 85 | <0.001* |
| Calprotectin (μg/ml) | | 18.5 | 74 | 74 | 82 | <0.001* |
| Composite markers | | any 2 or 3 markers ≧ cut-off | 85 | 77 | 86 | <0.001* |
| Spontaneous labor and delivery within 7 days | 38% (35/93) | | | | | |
| BPI (ng/ml) | | 1.2 | 83 | 84 | 88 | <0.001* |
| HNP 1-3 (ng/ml) | | 2.8 | 83 | 74 | 87 | <0.001* |
| Calprotectin (μg/ml) | | 15.2 | 80 | 62 | 81 | <0.001* |
| Composite markers | | any 2 or 3 markers ≧ cut-off | 83 | 81 | 87 | <0.001* |

MIAC, microbial invasion of the amniotic cavity; AF, amniotic fluid; BPI, bactericidal/permeability-increasing protein; HNP, human neutrophil defensins

TABLE 8
Table Diagnostic indices of various amniotic fluid antibacterial peptide concentrations and
other previously published markers (amniotic fluid Gram stain, white blood cell (WBC)
count ≧ 50 cells/ml and glucose concentration ≦ 14 mg/dl) for the identification of
microbial invasion of the amniotic cavity (n = 25) in patients with preterm labor and
intact membranes who had the results for every marker (n = 111)

| | Sensi- | | | | |
|---|---|---|---|---|---|
| | tivity | | Specificity | | Odds ratio with |
| Markers | % | n | % | n | 95% Cl |
| Gram stain positive | 44 | 11/25 | 99 | 85/86 | 66.7 (7.9-558.4) |
| Amniotic fluid WBC count ≧ 50 cells/ml | 68 | 17/25 | 92 | 79/86 | 23.9 (7.6-75.1) |
| Amniotic fluid glucose ≦ 14 mg/dl | 44 | 11/25 | 79 | 68/86 | 2.9 (1.2-7.6) |
| Gram stain positive plus amniotic fluid WBC count ≧ 50 cells/ml | 80 | 20/25 | 92 | 79/86 | 45.1 (12.9-157) |
| Gram stain positive plus aminotic fluid glucose ≦ 14 mg/dl | 60 | 15/25 | 79 | 68/86 | 5.7 (2.2-14.7) |
| Amniotic fluid WBC count ≧ 50 cells/ml plus glucose ≦ 14 mg/dl | 76 | 19/25 | 73 | 63/86 | 8.6 (3.0-24.4) |
| Gram stain positive plus amniotic fluid WBC count ≧ 50 cells/ml | 80 | 20/25 | 73 | 63/86 | 10.9 (3.7-32.6) |
| plus amniotic fluid glucose ≦ 14 mg/dl | | | | | |
| BPI ≧ 3.7 ng/ml | 80 | 20/25 | 79 | 68/86 | 15.1 (4.9-45.8) |
| HNP 1-3 ≧ 7.8 ng/ml | 80 | 20/25 | 79 | 68/86 | 15.1 (4.9-45.8) |
| Calprotectin ≧ 27.9 μg/ml | 80 | 20/25 | 78 | 67/86 | 14.1 (4.7-42.6) |
| Composite markers (any 2 or 3 antibacterial peptides with | 80 | 20/25 | 83 | 71/86 | 18.9 (6.1-58.4) |
| concentration ≧ cut-off) | | | | | |
| Gram stain positive plus amniotic fluid WBC count ≧ 50 cells/ml | 88 | 22/25 | 81 | 70/86 | 32.1 (8.5-120.5) |
| plus composite markers | | | | | |

BPI; bactericidal/permeability-increasingprotein; HNP, human neutrophil defensins; Cl confidence interval

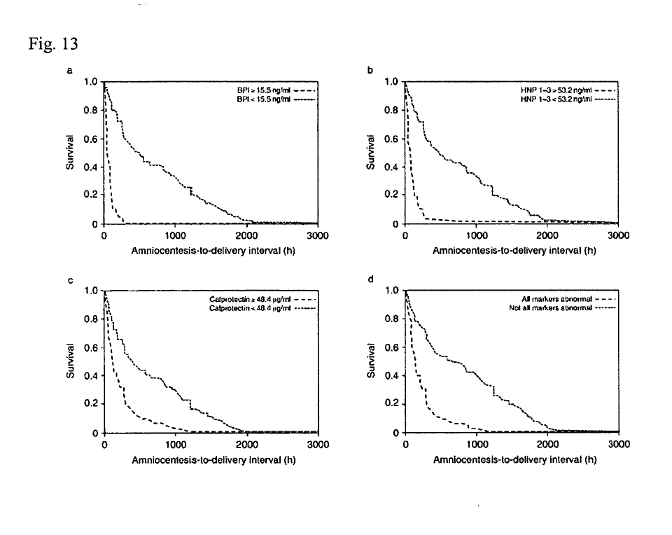

The interval to delivery was calculated to assess the relationship between intra-amniotic inflammation and the duration of the amniocentesis-to-delivery interval. Overall, as can be seen from Table 9, the amniocentesis-to-delivery interval was significantly shorter in subjects whose amniotic fluid concentrations of antibacterial peptides were above the cut-off values derived from ROC curves than those below the cut-off values. Cox proportional hazard model was used to examine the relationship between the duration of the amniocentesis-to-delivery interval and various antibacterial peptide concentrations in amniotic fluid, while adjusting for the status of amniotic fluid culture, gestational age and cervical dilatation at amniocentesis. Spontaneous labor and delivery was entered in the model as the event of interest. The exemplary cut-off values for BPI, HNP 1-3 and calprotectin were derived from the ROC curve analysis for the identification of subjects who had intra-amniotic fluid inflammation (amniotic fluid WBC count ³ 50 cells/ml). Each antibacterial peptide biomarker was entered in a separate model, as there was a high correlation in amniotic fluid among them. Table 10 displays the hazard ratio and 95% confidence intervals for the interval from amniocentesis to delivery according to amniotic fluid concentration of BPI, HNP 1-3 and calprotectin at the cut-off value used to identify intra-amniotic inflammation. Survival curves for various antibacterial peptides are displayed in FIG. 13. Among subjects with preterm labor and intact membranes who delivered within 72 h and whose placenta had been examined, 70% (23/33) had evidence of placental inflammation, while 30% (10/33) did not. All except one subject (95% (19/20)) who had amniotic fluid concentration of BPI≧15.5 ng/ml and HNP 1-3≧53.2 ng/ml and delivered within 72 h had evidence of inflammation in the extraplacental membranes and/or umbilical cord. Conversely, less than one-fifth (17% (4/23)) of subjects who delivered within 72 h with placental inflammation did not have elevated amniotic fluid concentrations of BPI and HNP 1-3 above these cut-off levels.

TABLE 9
Table Comparisons of interval from amniocentesis to delivery in patients who
had amniotic fluid antibacterial peptide concentration above the cut-off values
for the identification of intra-amniotic inflammation (white blood cell
count ≧ 50 cells/ml) and those who did not

| | | | Interval from | | |
|---|---|---|---|---|---|
| | | | amniocentesis | | |
| | | | to delivery (h) | | |
| | Cut-off values | n | Median | Range | p Value |
| BPI (ng/ml) | ≧15.5 | 27 | 14 | 2-651 | <0.001* |
| | <15.5 | 87 | 636 | 1.5-2904 | |
| HNP 1-3 (ng/ml) | ≧53.2 | 28 | 19 | 2-651 | <0.001* |
| | <53.2 | 86 | 675 | 1.5-2904 | |
| Calprotectin (μg/ml) | ≧48.4 | 28 | 24 | 2-840 | <0.001* |
| | <48.4 | 85 | 624 | 1.5-2904 | |
| Composite markers | all markers above the cut-off | 24 | 13 | 2-651 | <0.001* |
| | not all markers above the cut-off | 90 | 588 | 1.5-2904 | |

*Statistically significant, p < 0.05
BPI; bactericidal/permeability-increasing protein; HNP, human neutrophil defensins

TABLE 10
Table Hazard ratio of amniotic fluid concentration of antibacterial
peptides above the cut-off value used for the identification of
intra-amniotic inflammation (≧50 cells/ml) in relation to interval
from amniocentesis to delivery (h)

| | Beta | Wald | Hazard ratio | 95% CI |
|---|---|---|---|---|
| BPI (ng/ml) | | | | |
| BPI ≧ 15.5 ng/ml | 1.5 | 8.4 | 4.3 | 1.6-11.5 |
| MIAC | 0.7 | 2.3 | 2.0 | 0.8-5.0 |
| Gestational age (weeks) | 0.1 | 9.2 | 1.1 | 1.04-1.18 |
| Cervical dilatation (cm) | 0.3 | 13.5 | 1.4 | 1.2-1.7 |
| HNP 1-3 (ng/ml) | | | | |
| HNP 1-3 ≧ 53.2 ng/ml | 1.6 | 13.1 | 4.9 | 2.1-11.9 |
| MIAC | 0.6 | 2.0 | 1.9 | 0.8-4.4 |
| Gestational age (weeks) | 0.1 | 10.3 | 1.1 | 1.04-1.19 |
| Cervical dilatation (cm) | 0.3 | 14.6 | 1.4 | 1.2-1.7 |
| Calprotectin (μg/ml) | | | | |
| Calprotectin ≧ 48.4 μg/ml | 0.8 | 5.4 | 2.2 | 1.1-4.2 |
| MIAC | 1.2 | 11.9 | 3.4 | 1.7-6.9 |
| Gestational age (weeks) | 0.08 | 6.8 | 1.1 | 1.02-1.16 |
| Cervical dilatation (cm) | 0.3 | 12.2 | 1.4 | 1.2-1.6 |
| Composite markers | | | | |
| All markers ≧ cut-off | 1.4 | 7.6 | 3.9 | 1.5-10.1 |
| MIAC | 0.8 | 2.9 | 2.2 | 0.8-5.4 |
| Gestational age (weeks) | 0.1 | 8.5 | 1.1 | 1.03-1.18 |
| Cervical dilatation (cm) | 0.3 | 13.9 | 1.4 | 1.2-1.7 |

BPI, bactericidal/permeability-increasing protein;
MIAC, microbial invasion of the amniotic cavity;
HNP, human neutrophil defensins;
CI, confidence interval

These results demonstrate that MIAC, preterm parturition intra-amniotic inflammation, and PROM are associated with high amniotic fluid concentrations of the disclosed biomarkers HNP 1-3, BPI and calprotectin, and with each of them individually, or with combinations of them. Spontaneous labor at term is associated with a high amniotic fluid concentration of defensins, but not of BPI or calprotectin. Moreover, elevated amniotic fluid concentrations of BPI, HNP 1-3 and calprotectin are associated with intra-amniotic inflammation, histological chorioamnionitis and shorter amniocentesis-to-delivery interval in subjects presenting with preterm labor with intact membranes. In this example, immunoreactive BPI was detectable in approximately 10% of amniotic fluid samples obtained during the mid-trimester of pregnancy. In contrast, immunoreactive HNP 1-3 and calprotectin were detectable in virtually all samples in this example. These observations suggest that BPI is not constitutively present in mid-trimester amniotic fluid. This is the first report of increased amniotic fluid concentrations of BPI and calprotectin in subjects with MIAC. In the context of infection and/or inflammation, amniotic fluid WBCs may be the source of these antibacterial peptides. The significant positive correlation between amniotic fluid WBC count and the amniotic fluid concentration of each antimicrobial peptide examined in this example supports this concept. Previous studies have suggested that amniotic fluid WBCs are of fetal origin. Thus, it is possible that antimicrobial peptides in the amniotic fluid are, at least in part, of fetal origin. Other potential sources of antimicrobial peptides may be the leukocytes in the fetal lung, chorioamniotic membranes, placenta decidua, etc. There is no report, to date, regarding the expression of BPI and calprotectin in chorioamniotic membrane, placenta or decidua.

Elevated amniotic concentrations of BPI, HNP 1-3 and/or calprotectin, alone and in combination, are associated not only with MIAC, but also intra-amniotic inflammation, histological chorioamnionitis and shorter interval from amniocentesis to delivery in subjects with preterm labor and intact membranes.

An analysis of the data restricted to subjects with preterm labor and intact membranes shows that the antimicrobial peptides disclosed in this example have valuable diagnostic functions. The antimicrobial peptides had a sensitivity of 92% and a specificity of 93-98% in the identification of intra-amniotic inflammation (defined as an amniotic fluid WBC count above 50 cells/ml). Similarly, amniotic fluid antimicrobial peptide concentration was related to the detection of histological evidence of chorioamnionitis in subjects who delivered within 72 h of amniotic fluid retrieval. Ninety-five per cent (19/20) of subjects who had elevated HNP 1-3 (³ 53.2 ng/ml) or BPI (³ 15.5 ng/ml) and delivered within 72 h also had histological chorioamnionitis and/or funisitis.

Even in the absence of intra-amniotic inflammation, these antimicrobial peptide biomarkers predict preterm parturition. For example, nine out of ten subjects in this example who delivered within 72 h and showed no evidence of inflammation in the extraplacental membranes or umbilical cord had elevated amniotic concentrations of BPI or HNP 1-3, though these concentrations were below the cut-off level used in this study for the identification of intra-amniotic inflammation. Among subjects with preterm labor with intact membranes, those with amniotic HNP 1-3 of about 53.2 ng/ml or higher or BPI of about 15.5 ng/ml or higher had the highest risk for early spontaneous preterm delivery. Indeed, as shown in Table 10, the risk conferred by an elevation of antimicrobial peptides was higher than that of other factors, such as amniotic fluid culture positive for microorganisms, gestational age and even cervical dilatation at the time of amniocentesis.

Comparisons of previously published markers for the identification of MIAC (Gram stain, WBC count and glucose concentration in amniotic fluid) and the amniotic fluid concentration of the three antimicrobial peptides revealed that an amniotic fluid concentration of HNP 1-3 of about 3.7 ng/ml or greater, BPI of about 7.8 ng/ml or greater or calprotectin of about 27.9 mg/ml or greater, individually, had a sensitivity of 80% for the identification of MIAC, which can be seen in Table 7. The combined use of the composite antimicrobial peptide markers with Gram stain and amniotic fluid WBC count increased the sensitivity to 88%, with a specificity of 81% for the identification of MIAC.

In the absence of MIAC, both preterm parturition and preterm PROM were associated with increased amniotic fluid concentrations of defensins, BPI and calprotectin.

In sum, the disclosed biomarkers, either alone or in combinations of two or more, are detected in the amniotic fluid of subjects at risk for preterm complications, particularly those with MIAC and intra-amniotic inflammation, and elevated levels of the disclosed biomarkers in the amniotic fluid predicts the subjects' risk for such pre-term complications as impending preterm delivery and histological chorioamnionitis.

What is claimed is:

1. A diagnostic assay for detecting the presence of at least one biomarker indicative of intra-amniotic inflammation in a sample of amniotic fluid, comprising (A) mixing an adsorbent that binds at least one biomarker associated with intra-amniotic inflammation with a sample of amniotic fluid and then (B) monitoring said mixture for binding between said biomarker and said adsorbent, wherein said assay detects at least one biomarker that is a calgranulin.

2. A diagnostic assay as claimed in claim 1, wherein said adsorbent is an antibody immobilized on a solid substrate.

3. A diagnostic assay as claimed in claim 2, which is an ELISA.

4. A diagnostic assay as claimed in claim 2, wherein said solid substrate is a probe.

5. A diagnostic assay as claimed in claim 4, wherein said biomarker is detected by laser desorption/ionization mass spectrometry.

6. A diagnostic assay as claimed in claim 1, wherein said adsorbent is immobilized on a probe.

7. A diagnostic assay as claimed in claim 6, wherein said adsorbent is a hydrophobic adsorbent.

8. A diagnostic assay as claimed in claim 7, wherein said probe is a Ciphergen H4 probe or H50 probe.

9. A diagnostic assay as claimed in claim 1, which additionally tests for the presence of at least one defensin in said sample of amniotic fluid.

10. A diagnostic assay as claimed in claim 9, wherein said defensin is HNP-1 (alpha-defensin 1).

11. A diagnostic assay as claimed in claim 3, which additionally tests for the presence of at least defensin in said sample of amniotic fluid.

12. A diagnostic assay as claimed in claim 11, wherein said defensin is HNP-I (alpha-defensin 1).

13. A diagnostic assay as claimed in claim 5, which additionally tests for the presence of at least defensin in said sample of amniotic fluid.

14. A diagnostic assay as claimed in claim 13, wherein said defensin is HNP-1 (alpha-defensin 1).

15. A diagnostic assay as claimed in claim 1, wherein said calgranulin is calgranulin A.

16. A diagnostic assay as claimed in claim 1, wherein said calgranulin is calgranulin C.

17. A kit for detecting the presence of at least one biomarker indicative of intra-amniotic inflammation in a sample of amniotic fluid, comprising:
at least one adsorbent that binds at least one biomarker associated with intra-amniotic inflammation; and
instructions for mixing said adsorbent with a sample of amniotic fluid and monitoring said mixture for binding between said adsorbent and a biomarker in said sample,
wherein said kit includes at least one adsorbent that detects a calgranulin.

18. A kit as claimed in claim 17, wherein said adsorbent is an antibody is immobilized on a solid substrate.

19. A kit as claimed in claim 18, which additionally comprises an enzyme-antibody conjugate used to detect biomarker immobilized on said solid substrate.

20. A kit as claimed in claim 17, wherein said solid substrate is a probe.

21. A kit as claimed in claim 20, wherein said kit instructions specify analysis by laser desorption/ionization mass spectrometry.

22. A kit as claimed in claim 18, wherein said solid substrate is a probe.

23. A kit as claimed in claim 22, wherein said adsorbent is a hydrophobic adsorbent.

24. A kit as claimed in claim 23, wherein said probe is a Ciphergen H4 probe or H50 probe.

25. A kit as claimed in claim 17, additionally comprising at least one adsorbent that binds to at least one defensin.

26. A kit as claimed in claim 25, wherein said defensin is HNP-1 (alpha-defensin 1).

27. A kit as claimed in claim 19, which additionally comprising at least one adsorbent that binds to at least one defensin.

28. A kit as claimed in claim 27, wherein said defensin is HNP-1.

29. A kit as claimed in claim 21, which additionally comprising at least one adsorbent that binds to a defensin.

30. A kit as claimed in claim 29, wherein said defensin is HNP-1 (alpha-defensin 1).

31. A kit as claimed in claim 17, wherein said calgranulin is calgranulin A.

32. A kit as claimed in claim 17, wherein said calgranulin is C.

33. A method for qualifying the risk of preterm delivery in a pregnant subject, comprised of analyzing a sample of amniotic fluid from said subject for a level of at least one calgranulin.

34. A method according to claim 33, additionally comprising anaylzing said sample for the level of at least one defensin.

35. A method according to claim 33, wherein said calgranulin is calgranulin A or calgranulin C.

36. A method according to claim 32, wherein said defensin is HNP-1 (alpha-defensin 1) or HNP-2 (alpha-defensin 2).

37. A method according to claim 33, wherein said defensin is HNP-1 (alpha-defensin 1) or HNP-2 (alpha-defensin 2).

38. A method according to claim 37, wherein said defensin is HNP-1 (alpha-defensin 1).

39. A method for qualifying the risk of preterm delivery in a pregnant subject, comprising:
(A) providing a spectrum generated by subjecting a sample of amniotic fluid from said subject to mass spectroscopic analysis that includes profiling on a biologically- or chemically-derivatized affinity surface; and
(B) putting said spectrum through pattern-recognition analysis that is keyed to at least one peak indicative of the presence of a calgranulin in said sample.

40. A method according to claim 39, wherein said pattern-recognition analysis additionally is keyed to at least one peak indicative of a defensin.

41. A method according to claim 39, wherein said calgranulin is calgranulin A or calgranulin C.

42. A method according to claim 40, wherein said defensin is HNP-1 (alpha-defensin 1) or HNP-2 (alpha-defensin 2).

43. A method according to claim 42, wherein said defensin is HNP-1 (alpha-defensin 1).

44. A method according to claim 42, wherein said calgranulin is calgranulin A or calgranulin C.

45. A method according to claim 43, wherein said calgranulin is calgranulin A or calgranulin C.

46. A method according to claim 39, wherein said chemically-derivatized affinity surface is a Ciphergen H4 probe or H50 probe.

47. A method according to claim 39, wherein said subject does not have a white blood cell count that is elevated out of the normal range.

48. A method of identifying a subject at risk for a pre-term complication comprising detecting one or more biomarkers comprising a defensin, BPI, calprotectin, or a calgranulin in amniotic fluid from the subject.

49. The method of claim 48, wherein the one or more biomarkers detected comprises calprotectin or a calgranulin.

50. The method of claim 49, wherein the one or more biomarkers detected comprises a calgranulin.

51. The method of claim 50, wherein the calgranulin is calgranulin A or C.

52. The method of claim 51, wherin the calgranulin is calgranulin C.

53. The method of claim 49, wherein the one or more biomarkers detected comprises calprotectin.

54. The method of claim 48, wherein the one or more biomarkers detected comprises calprotectin or a calgranulin and one or more of a defensin or BPI.

55. The method of claim 54, wherein the one or more biomarkers detected comprises calprotectin and one or more of a defensin or BPI.

56. The method of claim 55, wherein the one or more biomarkers detected comprises calprotectin, a defensin, and BPI.

57. The method of claim 54, wherein the one or more biomarkers detected comprises a calgranulin and a defensin.

58. The method of claim 57, wherein the calgranulin is calgranulin A or C.

59. The method of claim 58, wherein the calgranulin is calgranulin C.

60. The method of claim 59, wherein the defensin is defensin 1.

61. The method of claim 59, wherein the defensin is defensin 2.

62. The method of claim 48, wherein the preterm complication comprises preterm parturition, preterm PROM, intra-amniotic inflammation, or MIAC.

63. The method of claim 62, wherein the preterm complication comprises preterm parturition.

64. The method of claim 62, wherein the preterm complication comprises preterm PROM.

65. The method of claim 62, wherein the preterm complication comprises intra-amniotic inflammation.

66. The method of claim 62, wherein the preterm complication comprises MIAC.

67. The method of claim 62, wherein detecting one or more biomarkers comprises determining that the concentration of one or more of defensin, BPI, or calprotectin is at or above a diagnostic concentration for defensin, BPI, or calprotectin.

68. The method of claim 67, wherein two or more of defensin, BPI, or calprotectin are at or above a diagnostic concentration for defensin, BPI, or calprotectin.

69. The method of claim 68, wherein defensin, BPI, and calprotectin are at or above a diagnostic concentration for defensin, BPI, or calprotectin.

70. The method of claim 67, wherein the diagnostic concentration for defensin is about 2 ng/ml or higher, the diagnostic concentration for BPI is about 2 ng/ml or higher, and the diagnostic concentration for calprotectin is about 12 ng/ml or higher.

71. The method of claim 70, wherein the diagnostic concentration for defensin is about 3 ng/ml or higher, the diagnostic concentration for BPI is about 2 ng/ml or higher, and the diagnostic concentration for calprotectin is about 15 ng/ml or higher.

72. The method of claim 71, wherein wherein the diagnostic concentration for defensin is about 3 ng/ml or higher, the diagnostic concentration for BPI is about 2 ng/ml or higher, and the diagnostic concentration for calprotectin is about 15 ng/ml or higher.

73. The method of claim 72, wherein wherein the diagnostic concentration for defensin is about 6 ng/ml or higher, the diagnostic concentration for BPI is about 3 ng/ml or higher, and the diagnostic concentration for calprotectin is about 20 ng/ml or higher.

74. The method of claim 73, wherein the preterm complication is MIAC.

75. The method of claim 74, wherein the diagnostic concentration for defensin is about 65 ng/ml or higher, the diagnostic concentration for BPI is about 28 ng/ml or higher, and the diagnostic concentration for calprotectin is about 25 ng/ml or higher.

76. The method of claim 75, wherein the preterm complication is preterm PROM and MIAC.

77. The method of claim 75, wherein the diagnostic concentration for defensin is about 45 ng/ml or higher, the diagnostic concentration for BPI is about 12 ng/ml or higher, and the diagnostic concentration for calprotectin is about 42 ng/ml or higher.

78. The method of claim 77, wherein the preterm complication is intra-amniotic inflammation.

79. The method of claim 48, wherein detecting the one or more biomarkers comprises using a binding agent to bind the one or more biomarkers in the amniotic fluid.

80. The method of claim 79, wherein detecting the one or more biomarkers further comprises using an immunoassay to determine the concentration of the one or more biomarkers.

81. The method of claim 80, wherein the immunoassay is ELISA.

82. A kit for detecting two or more of a defensin, BPI, calprotectin, or a calgranulin in amniotic fluid from a subject, comprising two or more binding agents, each adapted to bind to one of a defensin, BPI, calprotectin, or a calgranulin.
